(19) United States
(12) Patent Application Publication
(10) Pub. No.: US 2003/0175724 A1
(43) Pub. Date: Sep. 18, 2003

(54) Promoter libraries and their use in identifying promoters, transcription initiation sites and transcription factors

(76) Inventors: Wei Zhang, Houston, TX (US); Limei Hu, Pearland, TX (US); Stanley Hamilton, Bellaire, TX (US)

Correspondence Address: FULBRIGHT & JAWORSKI L.L.P., 600 CONGRESS AVENUE, SUITE 2400, AUSTIN, TX, 78701-3271, US

(21) Appl. No.: 10/132,660

(22) Filed: Apr. 25, 2002

Related U.S. Application Data
(60) Provisional application No. 60/287,221, filed on Apr. 27, 2001.

(51) Int. Cl. C12Q 1/68; G01N 33/53; C07H 21/04; C12P 19/34

(52) U.S. Cl. 435/6; 435/7.1; 435/91.2; 536/24.3

(57) ABSTRACT

The present invention provides methods for the identification of promoters and transcription initiation sites. More particularly, the invention provides for the production of a promoter library, and uses of the library in the identification of transcription factors that interact with previously unidentified promoter elements.

The present application claims priority to co-pending U.S. Provisional Patent Application Serial No. 60/287,221 filed on Apr. 27, 2001. The entire text of the above-referenced disclosure is specifically incorporated herein by reference without disclaimer.

### BACKGROUND OF THE INVENTION

A. Field of the Invention

The present invention relates to the fields of molecular biology and nucleic acid biochemistry. More particularly, the invention provides new methods for identification of promoters, transcription initiation sites and transcription factors.

B. Related Art

The rapid progress of the human genome project allows new strategies for the functional genomic analysis of normal and abnormal cells. The total number of expressed human genes has been estimated to be about 100,000, with about 11,000 genes being expressed in any particular cell type (Alberts et al., 1994). These genes can be grouped by their level of expression into abundant, intermediate abundant and rare abundant classes. These classes contain about 4-10 genes, 500 genes, and 11,000 genes respectively, comprising 10%, 40%, and 50% of the total transcripts (Alberts et al., 1994). The majority of expressed genes, therefore, belong to the rare abundant class. Most of the processes for gene identification also need to focus on this category.

Serial analysis of gene expression (SAGE) (U.S. Pat. No. 5,866,330) is based on the use of short (i.e., 9-10 base pair) nucleotide sequence tags that identify a defined position in an mRNA and are used to ascertain the identity of the corresponding transcript and gene. The cDNA tags are generated from mRNA samples, randomly paired, concatenated, cloned, and sequenced. While this method allows the analysis of a large number of transcripts, the identification of individual genes requires sequencing of tens of thousands of tags for comparison of even a small number of samples. Although SAGE provides a comprehensive picture of gene expression, it cannot be specifically directed at a small subset of the transcriptome (Zhang et al., 1997; Velculescu et al., 1995). Data on the most abundant transcripts is the easiest and fastest to obtain, while about a megabase of sequencing data is needed for confident analysis of low abundance transcripts. In addition, SAGE reveals no information about regulatory regions.

Gene expression is tightly regulated in both temporal and tissue specific fashions. Abnormal gene expression in pathological situations can alter the normal cellular behavior leading to various abnormalities such as neoplasia. Analysis of gene expression in various normal conditions can provide information regarding basic cell physiology. In pathological conditions, the abnormally expressed genes can serve as markers for early diagnosis, as targets for drug design, as indicators for treatment responsiveness, and for prognosis.

Over one million expressed sequence tags (EST) from the human genome are listed in the current NCBI dbEST database. Ultimately, most of the expressed genes from human genome will be indexed in the EST database. Maximal use of EST information will greatly accelerate the gene identification process, e.g., using an EST sequence to search the UniGene database to obtain the cluster information for that sequence and to obtain the original plasmids used for EST project for further analysis (Boguski, 1995; Gerhold and Caskey, 1996).

Equally as important to the understanding of gene expression, and the normal and pathologic states arising therefrom, is the examination of promoters. Promoter libraries have been generated using a “trapping” approach. Genomic DNA is inserted randomly into a “headless” expression vector, i.e., a promoterless construct that encodes a selectable or screenable marker protein such as luciferase or β-galactosidase. If the randomly inserted DNA can act as a promoter, the marker protein is expressed. A library can be constructed by selecting against those sequences that do not function as a promoter. Unfortunately, “trapping” libraries can miss out on important information.

Due to the large size of many genomes, powerful tools are required to probe their entire contents for elements such as promoters identification. Most of the methods currently used for whole genome level can be performed in only a few laboratories because they are either complicated or very costly. These include DNA microarray techniques (Lockhart et al., 1996; DeRisi et al., 1996), SAGE, or large-scale sequencing in the cancer genome anatomy project (CGAP) (Strausberg et al, 1997).

Many of these techniques also are limited in the information they can derive. For example, SAGE can miss relevant information due to the presence of repetitive elements, such as Alu sequences, that are located in the 3′ UTR. Poly-A sequences, possibly reflecting multiple genes, may be obscured in a SAGE analysis.

Thus, it is important to develop new, more efficient techniques to assist in gene expression profiling and in the rapid identification of promoters, as well as in defining the requirements for initiation of transcription. Transcription factors also are critical to an understanding of gene regulation, and the techniques should be amenable to modification for identifying these targets as well. Importantly, these techniques should avoid the known shortcomings of other techniques, such as SAGE.

### SUMMARY OF THE INVENTION

Thus, in accordance with the present invention, there is provided a method for generating a promoter library comprising:

(a) obtaining an RNA-containing composition from a cell;

(b) adding reverse transcriptase and a pair of primers to the composition, wherein the primers comprise

(i) an oligodT as a down-stream primer, and

(ii) a primer comprising three guanine residues at its 3-prime end as an up-stream primer, the primer also comprising a class II restriction enzyme site and a class III restriction enzyme site, wherein the class III site is 5′ to the class II site,

and incubating the primers and the reverse transcriptase under conditions supporting reverse transcription of a first corresponding cDNA strand and template switching by the reverse transcriptase;

(c) adding DNA polymerase to the product of step (b) under conditions supporting generation of a second corresponding cDNA;

(d) cleaving the cDNA population with a class III restriction enzyme that cleaves the up-stream primer generated class III restriction enzyme site;

(e) isolating the cDNA fragments lacking the poly-A tail of step (d), the fragments being designated as TIPS tags;

(f) ligating a linker to the TIPS tags;

(g) cleaving the TIPS tags+linkers with a class II restriction enzyme that cleaves the up-stream primer generated class II restriction site;

(h) obtaining the antisense strands from those portions of the TIP tags+linkers of step (g) that contain 5′ cDNA coding information;

(i) amplifying DNA sequences from genomic DNA using the antisense strands of step (h) and a random primer; and

(j) cloning the amplified products of step (i).

The method may further comprise amplification of the cDNA prior to step (d), such as DNA polymerase chain reaction. The class III restriction enzyme may be BsmF1, and the class II restriction enzyme may be selected from the group consisting of Hind III, EcoRI, SalI, BamHI and BssK I. The RNA composition may be poly-A RNA. The up-stream primer may further comprises a marker that permits isolation of the TIPS tags, for example, through binding of ligand (e.g., biotin).

Step (e) above may comprise binding of the biotin marker to streptavidin coated magnetic beads. It also may further comprise filling in the class III restriction enzyme site overhangs prior to step (f). It also may comprise, in step (j), cloning the amplified products up-stream of a reporter coding region to create a promoter-reporter library. The method may further comprise cloning the TIPS tag, or a fragment thereof.

The reporter coding region may be β-gal, luciferase or green fluorescent protein. The method may further comprise transforming a population of host cells with the promoter-reporter library, for example, bacterial cells. The method may further comprise screening the transformed bacteria cells for expression of the reporter, and further, sequencing expression positive clones.

In another embodiment, there is provided a method for identifying a transcription factor for a promoter comprising:

(a) obtaining an RNA-containing composition from a cell;

(b) adding reverse transcriptase and a pair of primers to the composition, wherein the primers comprise

(i) an oligodT as a down-stream primer, and

(ii) a primer comprising 3 guanine residues at its 3-prime end as an up-stream primer, the primer also comprising a class II restriction enzyme site and a class III restriction enzyme site, wherein the class III site is 5′ to the class II site, and incubating the primers and the reverse transcriptase under conditions supporting reverse transcription of a first corresponding cDNA strand and template switching by the reverse transcriptase;

(c) adding DNA polymerase to the product of step (b) under conditions supporting generation of a second corresponding cDNA;

(d) cleaving the cDNA population with a class III restriction enzyme that cleaves the up-stream primer generated class III restriction enzyme site;

(e) isolating the cDNA fragments lacking the poly-A tail of step (d), the fragments being designated as TIPS tags;

(f) ligating a linker to the TIPS tags;

(g) cleaving the TIPS tags+linkers with a class II restriction enzyme that cleaves the up-stream primer generated class II restriction site;

(h) obtaining the antisense strands from those portions of the TIP tags+linkers of step (g) that contain 5′ cDNA coding information;

(i) amplifying DNA sequences from genomic DNA using the antisense strands of step (h) and a random primer; and

(j) cloning the amplified products of step (i)

(k) sequencing the expression positive clones of step (j); and

(l) using the promoter identified in step (k) to identify a transcription factor acting thereon.

Step (l) may comprise co-transformation, into a population of host cells, of (i) a construct comprising a reporter coding region under the control of a promoter identified in step (k); and (ii) a construct comprising a cDNA expression vector, wherein expression of the reporter in the presence of a given cDNA, but not in the absence of the same cDNA, indicates that the cDNA encodes a transcription factor that acts on the promoter. The host cell population may comprise yeast cells. The method may further comprise sequencing of a cDNA found to encode a transcription factor. The the cDNA expression construct may be derived from the same organism as the promoter, or a different organism.

In yet an additional embodiment, there is provided a method for identifying the transcription initiation site of a gene comprising:

(a) obtaining an RNA-containing composition from a cell;

(b) adding reverse transcriptase and a pair of primers to the composition, wherein the primers comprise

(i) an oligodT as a down-stream primer, and

(ii) a primer comprising 3 guanine residues at its 3-prime end as an up-stream primer, the primer also comprising a class II restriction enzyme site and a class III restriction enzyme site, wherein the class III site is 5′ to the class II site, and incubating the primers and the reverse transcriptase under conditions supporting reverse transcription of a first corresponding cDNA strand and template switching by the reverse transcriptase;

(c) adding DNA polymerase to the product of step (b) under conditions supporting generation of a second corresponding cDNA;

(d) cleaving the cDNA population with a class III restriction enzyme that cleaves the up-stream primer generated class III restriction enzyme site;

(e) isolating the cDNA fragments lacking the poly-A tail of step (d), the fragments being designated as TIPS tags;

(f) ligating a linker to the TIPS tags, the linker comprising a primer sequence;

(g) cleaving the TIPS tags+linkers with a class II restriction enzyme that cleaves the up-stream primer generated class II restriction site;

(h) isolating that portion of the TIP tags+linkers of step (g) that contains 5′ cDNA coding information;

(i) treating the composition of step (h) with ligase to generate fragments that contain coding information from two different cDNAs, designated as DITags;

(j) cleaving the DITags of step (i) with the class II restriction enzyme that cleaves the up-stream primer generated class II restriction enzyme site, thereby releasing the DITtags;

(k) concatenating the DITtags;

(l) cloning the concatemers of step (k);

(m) sequencing the cloned concatemers of step (l); and

(n) comparing the sequence information of step (m) with at least one corresponding genomic sequence,

thereby identifying the transcription start site of at least one corresponding mRNA.

The method may further comprise amplification of the cDNA prior to step (d), for example, by polymerase chain reaction. The method may further comprise amplifying DITags prior to cleaving by the class II enzyme. The up-stream primer may further comprise a marker that permits isolation of the TIPS tags, such as by binding a ligand (e.g., biotin). Step (e) may comprise binding of the biotin marker to streptavidin coated magnetic beads.

The method may further comprising filling in the class III restriction enzyme site overhangs generated by step (d). The class II restriction enzyme may be selected from the group consisting of Hind III, EcoRI, SalI, BamHI and BssK I. The class III restriction enzyme may be BsmF1. The method also may further comprise amplifying genomic sequences using a plurality of different primer sequences generated from sequence information obtained by sequencing of the TIPS tags.

### BRIEF DESCRIPTION OF THE DRAWINGS

The following drawings form part of the present specification and are included to further demonstrate certain aspects of the present invention. The invention may be better understood by reference to these drawings and the detailed description presented below.

FIG. 1. Schematic for TIPS Production.

FIG. 2. Schematic for TIPS Promoter Library Construction.

### DETAILED DESCRIPTION OF THE INVENTION

To understand the gene expression pattern in cells under particular physiological and pathological conditions, the analysis must be performed at the genome scale. The EST project aims to collect expressed human sequences through screening many cDNA libraries from various sources (Boguski, 1995). At the same time, the Human Genome Project is drawing to a close. Between these two approaches, it is hoped that one can also identify many previously unknown promoters. However, the ability to identify promoters is still limited by the completeness of the EST library, and it is well known that methods for identifying EST's are limited in many ways.

A powerful tool for providing rapid, quantitative determination of the abundance and nature of transcripts corresponding to expressed genes is serial analysis of gene expression (SAGE). This method is based on the identification of and characterization of partial, defined sequences of transcripts corresponding to gene segments. These defined transcript sequence “tags” are markers for genes which are expressed in a cell, a tissue, or an extract, for example.

SAGE is based on several principles. First, a short nucleotide sequence tag (9 to 10 bp) contains sufficient information content to uniquely identify a transcript provided it is isolated from a defined position within the transcript. For example, a sequence as short as 9 bp can distinguish 262,144 transcripts (4⁹) given a random nucleotide distribution at the tag site, whereas estimates suggest that the human genome encodes about 80,000 to 200,000 transcripts (Fields et al., 1994). The size of the tag can be shorter for lower eukaryotes or prokaryotes, for example, where the number of transcripts encoded by the genome is lower. For example, a tag as short as 6-7 bp may be sufficient for distinguishing transcripts in yeast.

Second, random dimerization of tags allows a procedure for reducing bias (caused by amplification and/or cloning). Third, concatenation of these short sequence tags allows the efficient analysis of transcripts in a serial manner by sequencing multiple tags within a single vector or clone. As with serial communication by computers, wherein information is transmitted as a continuous string of data, serial analysis of the sequence tags requires a means to establish the register and boundaries of each tag. All of these principles may be applied independently, in combination, or in combination with other known methods of sequence identification.

Nonetheless, SAGE has a number of limitations, including bias, time and cost. In addition, it provides no direct information on the structure of regulatory elements, such as promoters. Much additional work, even more timely and costly, is required to derive such information.

A. The Present Invention

The present invention provides new and improved methods for identifying promoters and transcription start sites, generating promoter libraries, identifying transcription factors and profiling gene expression. The methods rely, in general, on obtaining sequence information at the very 5′ end of cDNAs from a given cell type. This information facilitates creation of a fixed primer which, when paired with an upstream random primer, permits amplification of the intervening sequence. This intervening sequence will contain both regulatory sequences, i.e., promoters, as well as the transcriptional start site for each promoter.

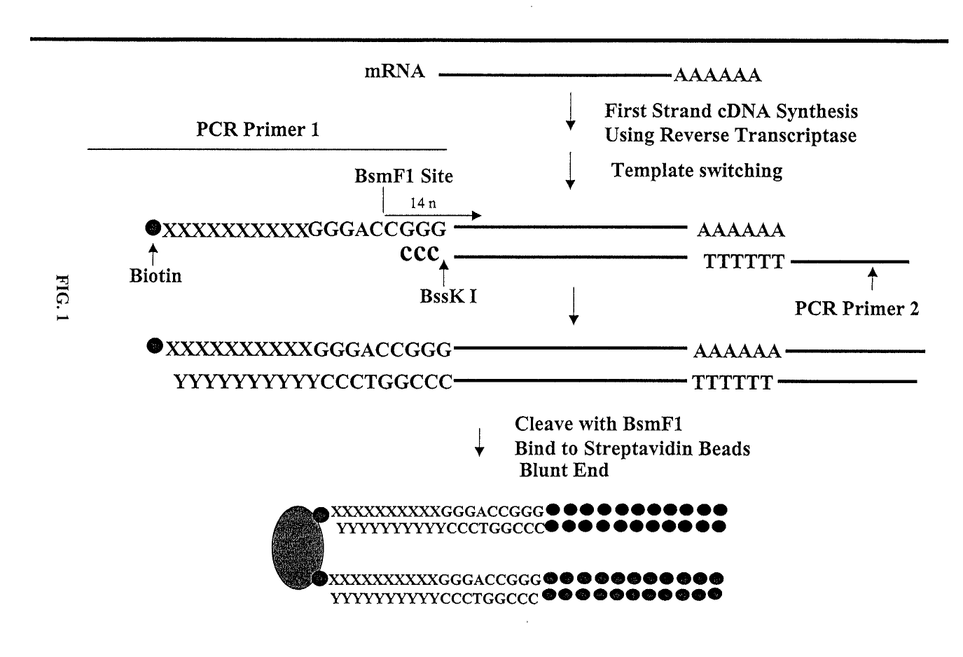

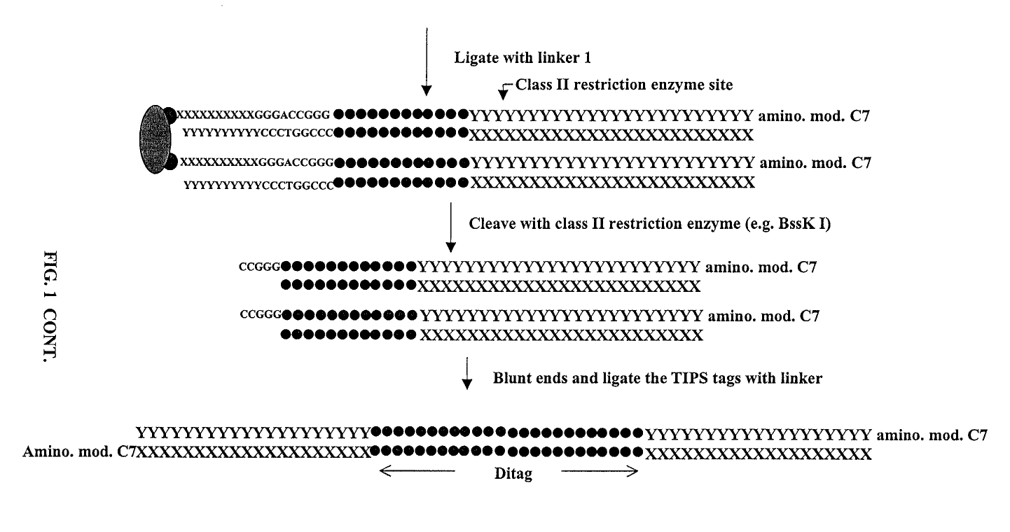

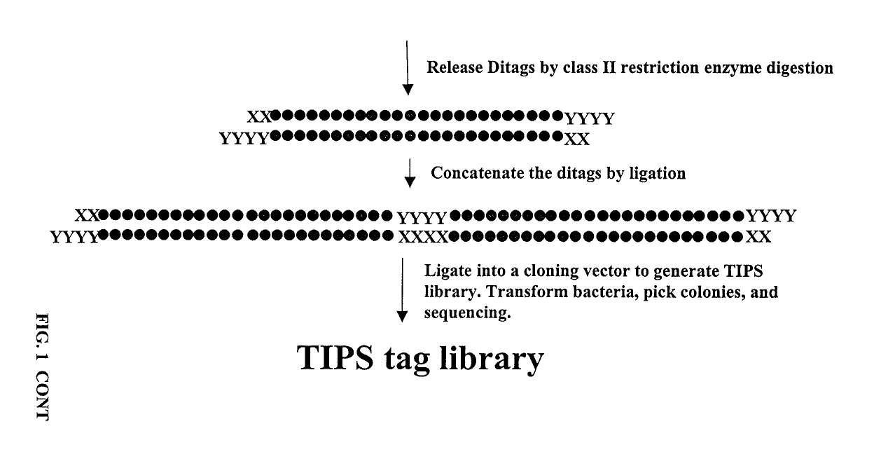

The invention starts with the isolation of RNA from cells. The RNA is then reverse transcribed into cDNA using a reverse transcriptase enzyme using a primer that binds to the poly-A tail of poly-A+ RNA (FIG. 1). Reverse transcriptase has the inherent property of adding multiple C's at the completion of the first strand synthesis. Playing off this characteristic, a second primer is designed that will hybridize to the poly-C stretch of the first DNA strand. This primer also has enzyme sites for a class III enzyme and another restriction site, with the class III site being closes to the coding region, i.e., closest to the poly-A site. cDNA synthesis (following “template switching”) is completed by generation of the second DNA strand.

Following second strand synthesis, the cDNA is digested with the class III enzyme, cutting between the poly-A sequences and the other enzyme site. This results in a cDNA fragment that contains the primer sequence and the 5′ sequences of the cDNA. The overhang generated by the class III enzyme is filled in to generate a blunt end, and then ligated to a linker. This linker may include yet another restriction site. The 5′-end fragment is then released using an enzyme that cleaves the other primer-generated restriction site (see above). Purification of the negative strand provides a suitable primer to further exploration of the corresponding genomic 5′ sequences.

In a further embodiment, the blunted molecule is ligated, not to a linker, but to other blunted molecules. This can be achieved while the blunted molecules are still attached to the bead to ensure proper orientation. Cleavages with the enzyme cleaving the other primer-generated site creates a “ditag,” which can be ligated with other ditags to generate a concatamer, which can then be cloned into a vector and multiple ditags sequenced at once.

Various aspects of the invention, including additional uses for the methods described above, are provided in detail in the following pages.

B. Serial Analysis of Gene Expression (SAGE)

SAGE provides for the detection of gene expression in a particular cell or tissue, or cell extract, for example, including at a particular developmental stage or in a particular disease state. The method comprises producing complementary deoxyribonucleic acid (cDNA) oligonucleotides, isolating a first defined nucleotide sequence tag from a first cDNA oligonucleotide and a second defined nucleotide sequence tag from a second cDNA oligonucleotide, linking the first tag to a first oligonucleotide linker, wherein the first oligonucleotide linker comprises a first sequence for hybridization of an amplification primer and linking the second tag to a second oligonucleotide linker, wherein the second oligonucleotide linker comprises a second sequence for hybridization of an amplification primer, and determining the nucleotide sequence of the tag(s), wherein the tag(s) correspond to an expressed gene.

FIG. 1 shows a schematic representation of the analysis of messenger RNA (mRNA) using SAGE as described in the method of the invention. mRNA is isolated from a cell or tissue of interest for in vitro synthesis of a double-stranded DNA sequence by reverse transcription of the mRNA. The double-stranded DNA complement of mRNA formed is referred to as complementary (cDNA).

The method further includes ligating the first tag linked to the first oligonucleotide linker to the second tag linked to the second oligonucleotide linker and forming a “ditag.” Each ditag represents two defined nucleotide sequences of at least one transcript, representative of at least one gene. Typically, a ditag represents two transcripts from two distinct genes. The presence of a defined cDNA tag within the ditag is indicative of expression of a gene having a sequence of that tag.

The analysis of ditags, formed prior to any amplification step, provides a means to eliminate potential distortions introduced by amplification, e.g., PCR. The pairing of tags for the formation of ditags is a random event. The number of different tags is expected to be large, therefore, the probability of any two tags being coupled in the same ditag is small, even for abundant transcripts. Therefore, repeated ditags potentially produced by biased standard amplification and/or cloning methods are excluded from analysis by the method of the invention.

The sequence is defined by cleavage with a first restriction endonuclease, and represents nucleotides either 5′ or 3′ of the first restriction endonuclease site, depending on which terminus is used for capture (e.g., 3′ when oligo-dT is used for capture as described herein).

The first endonuclease, termed “anchoring enzyme” or “AE” in FIG. 1, is selected by its ability to cleave a transcript at least one time and therefore produce a defined sequence tag from either the 5′ or 3′ end of a transcript. Preferably, a restriction endonuclease having at least one recognition site and therefore having the ability to cleave a majority of cDNAs is utilized. For example, as illustrated herein, enzymes which have a 4 base pair recognition site are expected to cleave every 256 base pairs (4⁴) on average while most transcripts are considerably larger. Restriction endonucleases which recognize a 4 base pair site include NlaIII. Other similar endonucleases having at least one recognition site within a DNA molecule (e.g., cDNA) will be known to those of skill in the art.

After cleavage with the anchoring enzyme, the most 5′ or 3′ region of the cleaved cDNA can then be isolated by binding to a capture medium. For example, streptavidin beads are used to isolate the defined 3′ nucleotide sequence tag when the oligo-dT primer for cDNA synthesis is biotinylated. Cleavage with the first or anchoring enzyme provides a unique site on each transcript which corresponds to the restriction site located closest to the poly-A tail. Likewise, the 5′ cap of a transcript (the cDNA) can be utilized for labeling or binding a capture means for isolation of a 5′ defined nucleotide sequence tag. Those of skill in the art will know other similar capture systems (e.g., biotin/streptavidin, digoxigenin/anti-digoxigenin) for isolation of the defined sequence tag as described herein.

SAGE is not limited to use of a single “anchoring” or first restriction endonuclease. It may be desirable to perform the method sequentially, using different enzymes on separate samples of a preparation, in order to identify a complete pattern of transcription for a cell or tissue. In addition, the use of more than one anchoring enzyme provides confirmation of the expression pattern obtained from the first anchoring enzyme. Therefore, the first or anchoring endonuclease may rarely cut cDNA such that few or no cDNA representing abundant transcripts are cleaved. Thus, transcripts which are cleaved represent “unique” transcripts. Restriction enzymes that have a 7-8 bp recognition site for example, would be enzymes that would rarely cut cDNA. Similarly, more than one tagging enzyme can be utilized in order to identify a complete pattern of transcription.

In one embodiment, the isolated defined nucleotide sequence tags are separated into two pools of cDNA, when the linkers have different sequences. Each pool is ligated via the anchoring, or first restriction endonuclease site to one of two linkers. When the linkers have the same sequence, it is not necessary to separate the tags into pools. The first oligonucleotide linker comprises a first sequence for hybridization of an amplification primer and the second oligonucleotide linker comprises a second sequence for hybridization of an amplification primer. In addition, the linkers further comprise a second restriction endonuclease site, also termed the “tagging enzyme” or “TE.” The method does not require, but preferably comprises, amplifying the ditag oligonucleotide after ligation.

The second restriction endonuclease cleaves at a site distant from or outside of the recognition site. For example, the second restriction endonuclease can be a class II restriction enzyme. Class II restriction endonucleases cleave at a defined distance up to 20 bp away from their asymmetric recognition sites (Szybalski, 1985). Examples of class II restriction endonucleases include BsmFI and Fok1. Other similar enzymes will be known to those of skill in the art (see below). The first and second “linkers” which are ligated to the defined nucleotide sequence tags are oligonucleotides having the same or different nucleotide sequences. Those of skill in the art can design such alternate linkers.

The linkers are designed so that cleavage of the ligation products with the second restriction enzyme, or tagging enzyme, results in release of the linker having a defined nucleotide sequence tag (e.g., 3′ of the restriction endonuclease cleavage site as exemplified herein). The defined nucleotide sequence tag may be from about 6 to 30 base pairs. Preferably, the tag is about 9 to 11 base pairs. Therefore, a ditag is from about 12 to 60 base pairs, and preferably from 18 to 22 base pairs.

The pool of defined tags ligated to linkers having the same sequence, or the two pools of defined nucleotide sequence tags ligated to linkers having different nucleotide sequences, are randomly ligated to each other “tail to tail.” The portion of the cDNA tag furthest from the linker is referred to as the “tail.” As illustrated in FIG. 1, the ligated tag pair, or ditag, has a first restriction endonuclease site upstream (5′) and a first restriction endonuclease site downstream (3′) of the ditag; a second restriction endonuclease cleavage site upstream and downstream of the ditag, and a linker oligonucleotide containing both a second restriction enzyme recognition site and an amplification primer hybridization site upstream and downstream of the ditag. In other words, the ditag is flanked by the first restriction endonuclease site, the second restriction endonuclease cleavage site and the linkers, respectively.

The ditag can be amplified by utilizing primers which specifically hybridize to one strand of each linker. Preferably, the amplification is performed by standard polymerase chain reaction (PCR) methods as described in U.S. Pat. No. 4,683,195. Alternatively, the ditags can be amplified by cloning in prokaryotic-compatible vectors or by other amplification methods known to those of skill in the art.

Cleavage of the amplified PCR product with the first restriction endonuclease allows isolation of ditags, which can be concatenated by ligation. After ligation, it may be desirable to clone the concatemers, although it is not required in the method of the invention. Analysis of the ditags or concatemers, whether or not amplification was performed, is by standard sequencing methods. Concatemers generally consist of about 2 to 200 ditags and preferably from about 8 to 20 ditags. While these are preferred concatemers, it will be apparent that the number of ditags which can be concatenated will depend on the length of the individual tags and can be readily determined by those of skill in the art without undue experimentation. After formation of concatemers, multiple tags can be cloned into a vector for sequence analysis, or alternatively, ditags or concatemers can be directly sequenced without cloning by methods known to those of skill in the art.

C. Primers and Probes

1. Primer Design

The term primer, as defined herein, is meant to encompass any nucleic acid that is capable of priming the synthesis of a nascent nucleic acid in a template-dependent process. Typically, primers are oligonucleotides from ten to twenty-five base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

According to the present invention, there are four different types of primers. The first two types of primers are illustrated in FIG. 1. Primer 2 is a reverse primer that primes synthesis of the first DNA strand of the cDNA. This is a “poly-dT” primer as it comprises multiple T residues. Primer 1 is a forward primer that primes synthesis of the positive (second) strand of the cDNA. It hybridizes to the first DNA strand by virtue of a poly-G stretch that is complementary to a poly-C stretch inserted by reverse transcriptase at the end of the first DNA strand synthesis.

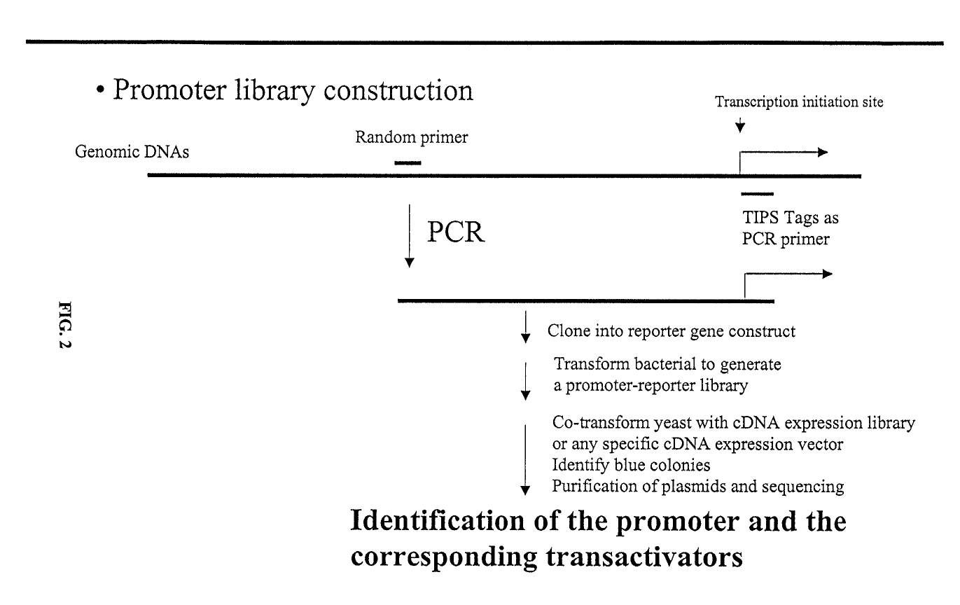

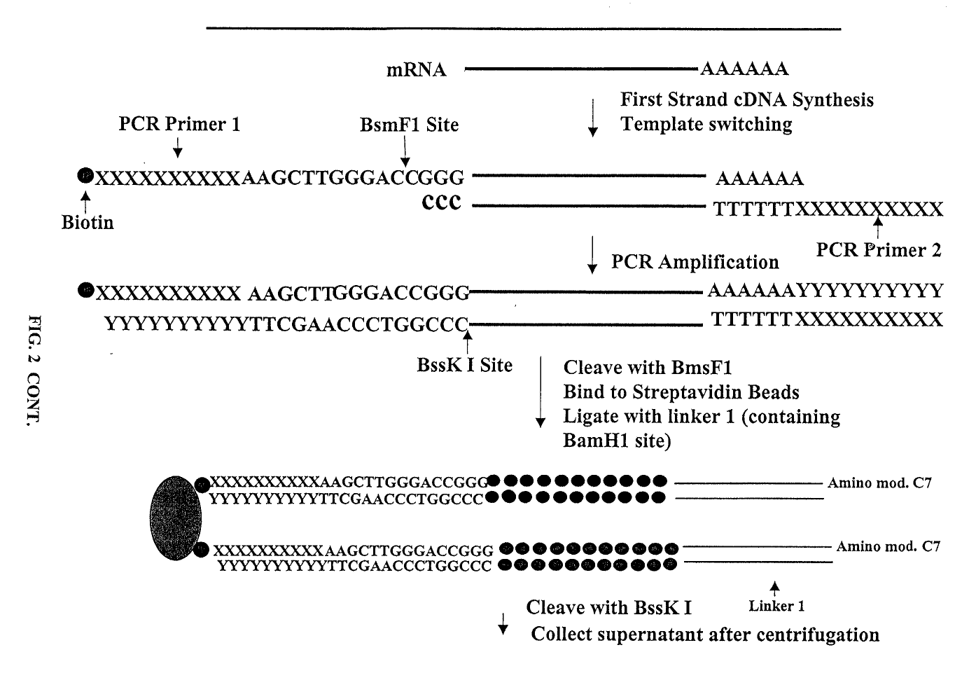

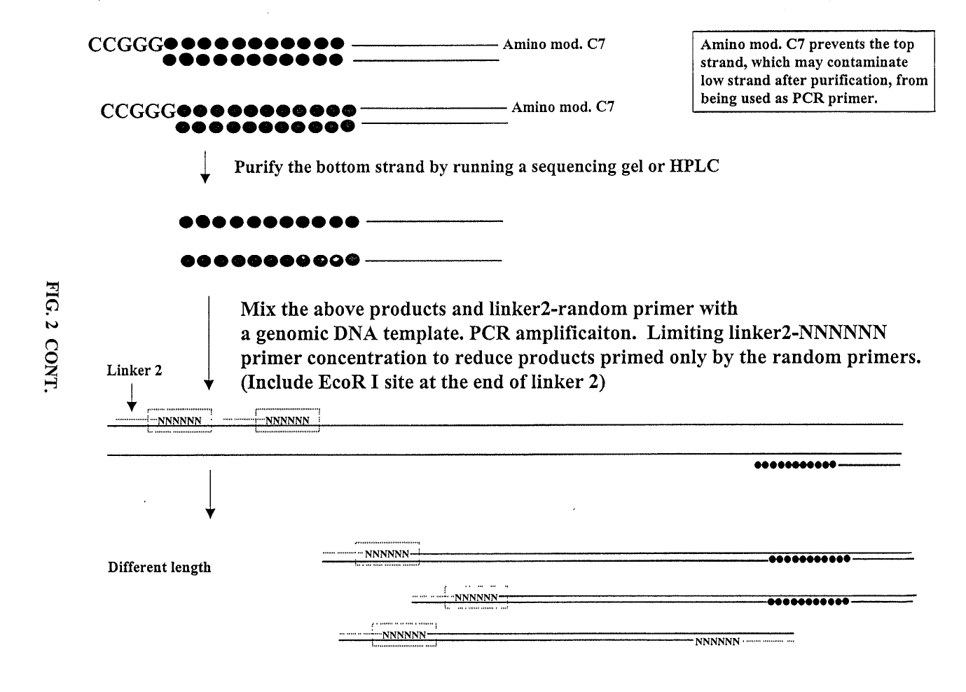

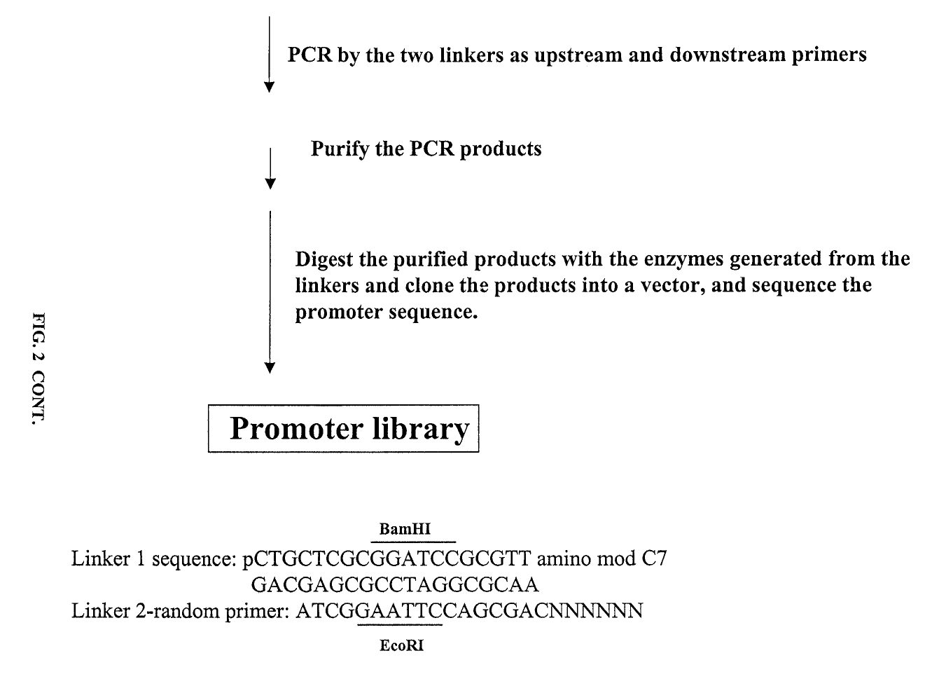

Two other primers are used in the generation of a promoter library. The first is a random primer that is used to bind to sequences 5′ to the promoter in genomic DNA. The second primer is derived from the DITags of the invention and primers at the 5′ end of the coding region. Use of these two primers together permits identification of intervening promoter sequences and, more generally, the production of promoter libraries. FIG. 2.

2. Hybridization

Suitable hybridization conditions will be well known to those of skill in the art. Typically, the present invention relies on high stringency conditions (low salt, high temperature), which are well known in the art. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

3. Oligonucleotide Synthesis

Oligonucleotide synthesis is performed according to standard methods. See, for example, Itakura and Riggs (1980). Additionally, U.S. Pat. No. 4,704,362; U.S. Pat. No. 5,221,619; U.S. Pat. No. 5,583,013; each describe various methods of preparing synthetic structural genes.

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference. Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers. (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore purification's are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligodeoxynucleotides (Gillam et al., 1978; Gillam et al., 1979). Under controlled conditions, polynucleotide phosphorylase adds predominantly a single nucleotide to a short oligodeoxynucleotide. Chromatographic purification allows the desired single adduct to be obtained. At least a trimer is required to start the procedure, and this primer must be obtained by some other method. The polynucleotide phosphorylase method works and has the advantage that the procedures involved are familiar to most biochemists.

Solid-phase methods. Drawing on the technology developed for the solid-phase synthesis of polypeptides, it has been possible to attach the initial nucleotide to solid support material and proceed with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, and Lyer, 1992) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

D. Polymerases

1. Reverse Transcriptases

According to the present invention, a variety of different reverse transcriptases may be utilized. The following are representative examples.

M-MLV Reverse Transcriptase. M-MLV (Moloney Murine Leukemia Virus Reverse Transcriptase) is an RNA-dependent DNA polymerase requiring a DNA primer and an RNA template to synthesize a complementary DNA strand. The enzyme is a product of the pol gene of M-MLV and consists of a single subunit with a molecular weight of 71 kDa. M-MLV RT has a weaker intrinsic RNaseH activity than Avian Myeloblastosis Virus (AMV) reverse transcriptase which is important for achieving long full-length complementary DNA (>7 kB).

M-MLV can be use for first strand cDNA synthesis and primer extensions. Storage recommend at −20° C. in 20 mM Tris-HCl (pH 7.5), 0.2M NaCl, 0.1 mM EDTA, 1 mM DTT, 0.01% Nonidet® P-40, 50% glycerol. The standard reaction conditions are 50 mM Tris-HCl (pH 8.3), 7 mM MgCl₂, 40 mM KCl, 10 mM DTT, 0.1 mg/ml BSA, 0.5 mM ³H-dTTP, 0.025 mM oligo(dT)₅₀, 0.25 mM poly(A)400 at 37° C.

M-MLV Reverse Transcriptase, RNase H Minus. This is a form of Moloney murine leukemia virus reverse transcriptase (RNA-dependent DNA polymerase) which has been genetically altered to remove the associated ribonuclease H activity (Tanese and Goff, 1988). It can be used for first strand cDNA synthesis and primer extension. Storage is at 20° C. in 20 mM Tris-HCl (pH 7.5), 0.2M NaCl, 0.1 mM EDTA, 1 mM DTT, 0.01% Nonidet® P-40, 50% glycerol.

AMV Reverse Transcriptase. Avian Myeloblastosis Virus reverse transcriptase is a RNA dependent DNA polymerase that uses single-stranded RNA or DNA as a template to synthesize the complementary DNA strand (Houts et al., 1979). It has activity at high temperature (42° C.-50° C.). This polymerase has been used to synthesize long cDNA molecules.

Reaction conditions are 50 mM Tris-HCl (pH 8.3), 20 mM KCl, 10 mM MgCl₂, 500 μM of each dNTP, 5 mM dithiothreitol, 200 μg/ml oligo-dT₍₁₂₋₁₈₎, 250 μg/ml polyadenylated RNA, 6.0 pMol ³²P-dCTP, and 30 U enzyme in a 7 μl volume. Incubate 45 min at 42° C. Storage buffer is 200 mM KPO₄ (pH 7.4), 2 mM dithiothreitol, 0.2% Triton X-100, and 50% glycerol. AMV may be used for first strand cDNA synthesis, RNA or DNA dideoxy chain termination sequencing, and fill-ins or other DNA polymerization reactions for which Klenow polymerase is not satisfactory (Maniatis et al., 1976).

2. DNA Polymerases

The present invention also contemplates the use of various DNA polymerase. Exemplary polymerases are described below.

Bst DNA Polymerase, Large Fragment. Bst DNA Polymerase Large Fragment is the portion of the Bacillus stearothermophilus DNA Polymerase protein that contains the 5′→3′ polymerase activity, but lacks the 5′→3′ exonuclease domain. BST Polymerase Large Fragment is prepared from an E. coli strain containing a genetic fusion of the Bacillus stearothermophilus DNA Polymerase gene, lacking the 5′→3′ exonuclease domain, and the gene coding for E coli maltose binding protein (MBP). The fusion protein is purified to near homogeneity and the MBP portion is cleaved off in vitro. The remaining polymerase is purified free of MBP (Iiyy et al., 1991).

Bst DNA polymerase can be used in DNA sequencing through high GC regions (Hugh & Griffin, 1994; McClary et al., 1991) and Rapid Sequencing from nanogram amounts of DNA template (Mead et al., 1991). The reaction buffer is 1×ThermoPol Butter (20 mM Tris-HCl (pH 8.8 at 25° C.), 10 mM KCl, 10 mM (NH₄)₂SO₄, 2 mM MgSO₄, 0.1% Triton X-100). Supplied with enzyme as a 10×concentrated stock.

Bst DNA Polymerase does not exhibit 3′→5′ exonuclease activity. 100 μ/ml BSA or 0.1% Triton X-100 is required for long term storage. Reaction temperatures above 70° C. are not recommended. Heat inactivated by incubation at 80° C. for 10 min. Bst DNA Polymerase cannot be used for thermal cycle sequencing. Unit assay conditions are 50 mM KCl, 20 mM Tris-HCl (pH 8.8), 10 mM MgCl₂, 30 nM M13mp18 ssDNA, 70 nM M13 sequencing primer (−47) 24 mer (NEB #1224), 200 μM daTP, 200 μM dCTP, 200 μM dGTP, 100 μM ³H-dTTP, 100 μg/ml BSA and enzyme. Incubate at 65° C. Storage buffer is 50 mM KCl, 10 mM Tris-HCl (pH 7.5), 1 mM dithiothreitol, 0.1 mM EDTA, 0.1% Triton-X-100 and 50% glycerol. Storage is at −20° C.

VENT_{R}® DNA Polymerase and VENT_{R}® (exo⁻) DNA Polymerase. Vent_{R} DNA Polymerase is a high-fidelity thermophilic DNA polymerase. The fidelity of Vent_{R} DNA Polymerase is 5-15-fold higher than that observed for Taq DNA Polymerase (Mattila et al., 1991; Eckert and Kunkel, 1991). This high fidelity derives in part from an integral 3′→5′ proofreading exonuclease activity in Vent_{R} DNA Polymerase (Mattila et al., 1991; Kong et al., 1993). Greater than 90% of the polymerase activity remains following a 1 h incubation at 95° C.

Vent_{R} (exo-) DNA Polymerase has been genetically engineered to eliminate the 3′→5′ proofreading exonuclease activity associated with Vent_{R} DNA Polymerase (Kong et al., 1993). This is the preferred form for high-temperature dideoxy sequencing reactions and for high yield primer extension reactions. The fidelity of polymerization by this form is reduced to a level about 2-fold higher than that of Taq DNA Polymerase (Mattila et al., 1991; Eckert & Kunkel, 1991). Vent_{R} (exo-) DNA Polymerase is an excellent choice for DNA sequencing and is included in our CircumVent Sequencing Kit (see pages 118 and 121).

Both Vent_{R} and Vent_{R} (exo-) are purified from strains of E. coli that carry the Vent DNA Polymerase gene from the archaea Thermococcus litoralis (Perler et al., 1992). The native organism is capable of growth at up to 98° C. and was isolated from a submarine thermal vent. They are useful in primer extension, thermal cycle sequencing and high temperature dideoxy-sequencing.

DEEP VENT_{R}™ DNA Polymerase and DEEP VENT_{R}™ (exo⁻) DNA Polymerase. Deep Vent_{R} DNA Polymerase is the second high-fidelity thermophilic DNA polymerase available from New England Biolabs. The fidelity of Deep Vent_{R} DNA Polymerase is derived in part from an integral 3′→5′ proofreading exonuclease activity. Deep Vent_{R} is even more stable than Vent_{R} at temperatures of 95 to 100° C.

Deep Vent_{R} (exo-) DNA Polymerase has been genetically engineered to eliminate the 3′→5′ proofreading exonuclease activity associated with Deep Vent_{R} DNA Polymerase. This exo- version can be used for DNA sequencing but requires different dNTP/ddNTP ratios than those used with Vent_{R} (exo-) DNA Polymerase. Both Deep Vent_{R} and Deep Vent_{R} (exo-) are purified from a strain of E. coli that carries the Deep Vent_{R} DNA Polymerase gene from Pyrococcus species GB-D (Perler et al., 1996). The native organism was isolated from a submarine thermal vent at 2010 meters (Jannasch et al., 1992) and is able to grow at temperatures as high as 104° C. Both enzymes can be used in primer extension, thermal cycle sequencing and high temperature dideoxy-sequencing.

T7 DNA Polymerase (unmodified). T7 DNA polymerase catalyzes the replication of T7 phage DNA during infection. The protein dimer has two catalytic activities: DNA polymerase activity and strong 3′→5′ exonuclease (Hori et al., 1979; Engler et al., 1983; Nordstrom et al., 1981). The high fidelity and rapid extension rate of the enzyme make it particularly useful in copying long stretches of DNA template.

T7 DNA Polymerase consists of two subunits: T7 gene 5 protein (84 kilodaltons) and E. coli thioredoxin (12 kilodaltons) (Hori et al., 1979; Studier et al., 1990; Grippo & Richardson, 1971; Modrich & Richardson, 1975; Adler & Modrich, 1979). Each protein is cloned and overexpressed in a T7 expression system in E. coli (Studier et al., 1990). It can be used in second strand synthesis in site-directed mutagenesis protocols (Bebenek & Kunkel, 1989).

The reaction buffer is 1×T7 DNA Polymerase Buffer (20 mM Tris-HCl (pH 7.5), 10 mM MgCl₂, 1 mM dithiothreitol). Supplement with 0.05 mg/ml BSA and dNTPs. Incubate at 37° C. The high polymerization rate of the enzyme makes long incubations unnecessary. T7 DNA Polymerase is not suitable for DNA sequencing.

Unit assay conditions are 20 mM Tris-HCl (pH 7.5), 10 mM MgCl₂, 1 mM dithiothreitol, 0.05 mg/ml BSA, 0.15 mM each dNTP, 0.5 mM heat denatured calf thymus DNA and enzyme. Storage conditions are 50 mM KPO₄ (pH 7.0), 0.1 mM EDTA, 1 mM dithiothreitol and 50% glycerol. Store at −20° C.

DNA Polymerase I (E. coli). DNA Polymerase I is a DNA-dependent DNA polymerase with inherent 3′→5′ and 5′→3′ exonuclease activities (Lehman, 1981). The 5′→3′ exonuclease activity removes nucleotides ahead of the growing DNA chain, allowing nick-translation. It is isolated from E. coli CM 5199, a lysogen carrying λpolA transducing phage (obtained from N. E. Murray) (Murray & Kelley, 1979). The phage in this strain was derived from the original polA phage encoding wild-type Polymerase I.

Applications include nick translation of DNA to obtain probes with a high specific activity (Meinkoth and Wahl, 1987) and second strand synthesis of cDNA (Gubler & Hoffmann, 1983; D'Alessio & Gerard, 1988). The reaction buffer is E. coli Polymerase I/Klenow Buffer (10 mM Tris-HCl (pH 7.5), 5 mM MgCl₂, 7.5 mM dithiothreitol). Supplement with dNTPs.

DNase I is not included with this enzyme and must be added for nick translation reactions. Heat inactivation is for 20 min at 75° C. Unit assay conditions are 40 mM KPO₄ (pH 7.5), 6.6 mM MgCl₂, 1 mM 2-mercaptoethanol, 20 μM dAT copolymer, 33 μM dATP and 33 μM ³H-dTTP. Storage conditions are 0.1 M KPO₄ (pH 6.5), 1 mM dithiothreitol, and 50% glycerol. Store at −20° C.

DNA Polymerase I, Large (Klenow) Fragment. Klenow fragment is a proteolytic product of E. coli DNA Polymerase I which retains polymerization and 3′→5′ exonuclease activity, but has lost 5′→3′ exonuclease activity. Klenow retains the polymerization fidelity of the holoenzyme without degrading 5′ termini.

A genetic fusion of the E. coli pola gene, that has its 5′→3′ exonuclease domain genetically replaced by maltose binding protein (MBP). Klenow Fragment is cleaved from the fusion and purified away from MBP. The resulting Klenow fragment has the identical amino and carboxy termini as the conventionally prepared Klenow fragment.

Applications include DNA sequencing by the Sanger dideoxy method (Sanger et al, 1977), fill-in of 3′ recessed ends (Sambrook et al., 1989), second-strand cDNA synthesis, random priming labeling and second strand synthesis in mutagenesis protocols (Gubler, 1987)

Reactions conditions are 1×E. coli Polymerase I/Klenow Buffer (10 mM Tris-HCl (pH 7.5), 5 mM MgCl2, 7.5 mM dithiothreitol). Supplement with dNTPs (not included). Klenow fragment is also 50% active in all four standard NEBuffers when supplemented with dNTPs. Heat inactivated by incubating at 75° C. for 20 min. Fill-in conditions: DNA should be dissolved, at a concentration of 50 g/ml, in one of the four standard NEBuffers (1×) supplemented with 33 μM each dNTP. Add 1 unit Klenow per μg DNA and incubate 15 min at 25° C. Stop reaction by adding EDTA to 10 MM final concentration and heating at 75° C. for 10 min. Unit assay conditions 40 mM KPO₄ (pH 7.5), 6.6 mM MgCl2, 1 mM 2-mercaptoethanol, 20 μM dAT copolymer, 33 μM dATP and 33 PM ³H-dTTP. Storage conditions are 0.1 M KPO₄ (pH 6.5), 1 mM dithiothreitol, and 50% glycerol. Store at −20° C.

Klenow Fragment (3′→5′ exo⁻). Klenow Fragment (3′→5′ exo-) is a proteolytic product of DNA Polymerase I which retains polymerase activity, but has a mutation which abolishes the 3′→5′ exonuclease activity and has lost the 5′→3′ exonuclease (Derbyshire et al., 1988).

A genetic fusion of the E. coli polA gene, that has its 3′→5′ exonuclease domain genetically altered and 5′→3′ exonuclease domain replaced by maltose binding protein (MBP). Klenow Fragment exo- is cleaved from the fusion and purified away from MBP. Applications include random priming labeling, DNA sequence by Sanger dideoxy method (Sanger et al., 1977), second strand cDNA synthesis and second strand synthesis in mutagenesis protocols (Gubler, 1987).

Reaction buffer is 1×E. coli Polymerase I/Klenow Buffer (10 mM Tris-HCl (pH 7.5), 5 mM MgCl₂, 7.5 mM dithiothreitol). Supplement with dNTPs. Klenow Fragment exo- is also 50% active in all four standard NEBuffers when supplemented with dNTPs. Heat inactivated by incubating at 75° C. for 20 min. When using Klenow Fragment (3′→5′ exo-) for sequencing DNA using the dideoxy method of Sanger et al. (1977), an enzyme concentration of 1 unit/5 μl is recommended.

Unit assay conditions are 40 mM KPO₄ (pH 7.5), 6.6 mM MgCl₂, 1 mM 2-mercaptoethanol, 20 μM dAT copolymer, 33 μM dATP and 33 μM ³H-dTTP. Storage conditions are 0.1 M KPO₄ (pH 7.5), 1 mM dithiothreitol, and 50% glycerol. Store at −20° C.

T4 DNA Polymerase. T4 DNA Polymerase catalyzes the synthesis of DNA in the 5′→3′ direction and requires the presence of template and primer. This enzyme has a 3′→5′ exonuclease activity which is much more active than that found in DNA Polymerase I. Unlike E. coli DNA Polymerase I, T4 DNA Polymerase does not have a 5′→3′ exonuclease function.

Purified from a strain of E. coli that carries a T4 DNA Polymerase overproducing plasmid. Applications include removing 3′ overhangs to form blunt ends (Tabor & Struhl, 1989; Sambrook et al., 1989), 5′ overhang fill-in to form blunt ends (Tabor & Struhl, 1989; Sambrook et al., 1989), single strand deletion subcloning (Dale et al., 1985), second strand synthesis in site-directed mutagenesis (Kunkel et al., 1987), and probe labeling using replacement synthesis (Tabor & Struhl, 1989; Sambrook et al., 1989).

The reaction buffer is 1×T4 DNA Polymerase Buffer (50 mM NaCl, 10 mM Tris-HCl, 10 mM MgCl₂, 1 mM dithiothreitol (pH 7.9 at 25° C.)). Supplement with 40 μg/ml BSA and dNTPs (not included in supplied 10×buffer). Incubate at temperature suggested for specific protocol.

It is recommended to use 100 μM of each dNTP, 1-3 units polymerase/μg DNA and incubation at 12° C. for 20 min in the above reaction buffer (Tabor & Struhl, 1989; Sambrook et al., 1989). Heat inactivated by incubating at 75° C. for 10 min. T4 DNA Polymerase is active in all four standard NEBuffers when supplemented with dNTPs.

Unit assay conditions are 50 mM NaCl, 10 mM Tris-HCl, 10 MM MgCl₂, 1 mM dithiothreitol (pH 7.9 at 25° C.), 33 μM dATP, dCTP and dGTP, 33 μM ³H dTTP, 70 μg/ml denatured calf thymus DNA, and 170 μg/ml BSA. Note: These are not suggested reaction conditions; refer to Reaction Buffer. Storage conditions are 100 mM KPO₄ (pH 6.5), 10 mM 2-mercaptoethanol and 50% glycerol. Store at −20° C.

E. Other Enzymes

1. Ligases

The following ligases are suitable for use in the present invention:

E. coli DNA Ligase. This enzyme catalyzes the formation of a phosphodiester bond in the presence of β-NAD between double-stranded DNA with 3′ hydroxyl and 5′ phosphate cohesive termini. Single-stranded DNA is not a substrate. Unit definition is defined as that amount of enzyme to give 50% lgation of HindIII digested λ DNA in 30 min at 16° C. in a final volume of 20 μl containing a 5′ termini concentration of 0.12 μM (300 μg/ml). Unit reaction conditions are 18.8 mM Tris-HCl (pH 8.3), 90.6 mM KCl, 4.6 MM MgCl₂, 3.8 mM DTT, 0.15 mM α-NAD, 10 MM (NH₄)₂SO₄ in 20 μl for 1 hr at 16° C.

T4 DNA Ligase. This ligase forms phosphodiester bonds in the presence of ATP between double-stranded DNA with 3′ hydroxyl nd 5′ phosphate termini. Single-stranded DNA is not a substrate. Unit reaction condition catalyzes the exchange of 1 mmol of ³²P-labeled pyrophosphate into ATP in 20 min at 37° C. Unit reaction conditions are 66 mM Tris-HCl (pH 7.6), 6.6 MM MgCl₂, 10 mM DTT, 66 μM ATP, 3.3 μM pyrophosphate and enzyme in 0.1 ml for 20 min at 3° C.

2. Restriction Enzymes

Restriction-enzymes recognize specific short DNA sequences of four to eight nucleotides long and cleave the DNA at a site within or near this sequence. The list below exemplifies the currently known restriction enzymes that may be used in the invention.

| | |
|---|---|
| Enzyme Name | Recognition Sequence |

| | |
|---|---|
| AatII | GACGTC |
| Acc65 I | GGTACC |
| Acc I | GTMKAC |
| Aci I | CCGC |
| Acl I | AACGTT |
| Afe I | AGCGCT |
| Afl II | CTTAAG |
| Afl III | ACRYGT |
| Age I | ACCGGT |
| Ahd I | GACNNNNNGTC |
| Alu I | AGCT |
| Alw I | GGATC |
| AlwN I | CAGNNNCTG |
| Apa I | GGGCCC |
| ApaL I | GTGCAC |
| Apo I | RAATTY |
| Asc I | GGCGCGCC |
| Ase I | ATTAAT |
| Ava I | CYCGRG |
| Ava II | GGWCC |
| Avr II | CCTAGG |
| Bae I | NACNNNNGTAPyCN |
| BamH I | GGATCC |
| Ban I | GGYRCC |
| Ban II | GRGCYC |
| Bbs I | GAAGAC |
| Bbv I | GCAGC |
| BbvC I | CCTCAGC |
| Bcg I | CGANNNNNNTGC |
| BciV I | GTATCC |
| Bcl I | TGATCA |
| Bfa I | CTAG |
| Bgl I | GCCNNNNNGGC |
| Ggl II | AGATCT |
| Blp I | GCTNAGC |
| Bmr I | ACTGGG |
| Bpm I | CTGGAG |
| BsaA I | YACGTR |
| BsaA I | GATNNNNATC |
| BsaH I | GRCGYC |
| Bsa I | GGTCTC |
| BsaJ I | CCNNGG |
| BsaW I | WCCGGW |
| BseR I | GAGGAG |
| Bsg I | GTGCAG |
| BsiE I | CGRYCG |
| BsiHKA I | GWGCWC |
| BsiW I | CGTACG |
| Bsl I | CCNNNNNNNGG |
| BsmA I | GTCTC |
| BsmB I | CGTCTC |
| BsmF I | GGGAC |
| Bsm I | GAATGC |
| BsoB I | CYCGRG |
| Bspl286 I | GDGCHC |
| BspD I | ATCGAT |
| BspE I | TCCGGA |
| BspH I | TCATGA |
| BspM I | ACCTGC |
| BsrB I | CCGCTC |
| BsrD I | GCAATG |
| BsrF I | RCCGGY |
| BsrG I | TGTACA |
| Bsr I | ACTGG |
| BssH II | GCGCGC |
| BssK I | CCNGG |
| Bst4C I | ACNGT |
| BssS I | CACGAG |
| BstAp I | GCANNNNNTGC |
| BstB I | TTCGAA |
| BstE II | GGTNACC |
| BstF5 I | GGATGNN |
| BstN I | CCWGG |
| BstU I | CGCG |
| BstX I | CCANNNNNNTGG |
| BstY I | RGATCY |
| BstZ17 I | GTATAC |
| Bsu36 I | CCTNAGG |
| Btg I | CCPuPyGG |
| Btr I | CACGTG |
| Cac8 I | GCNNGC |
| Cla I | ATCGAT |
| Dda I | CTNAG |
| Dpn I | GATC |
| Dpn II | GATC |
| Dra I | TTTAAA |
| Dra III | CACNNNGTG |
| Drd I | GACNNNNNNGTC |
| Eae I | YGGCCR |
| Eag I | CGGCCG |
| Ear I | CTCTTC |
| Eci I | GGCGGA |
| EcoN I | CCTNNNNNAGG |
| EcoO109 I | RGGNCCY |
| EcoR I | GAATTC |
| EcoR V | GATATC |
| Fau I | CCCGCNNNN |
| Fnu4H I | GCNGC |
| Fok I | GGATG |
| Fse I | GGCCGGCC |
| Fsp I | TGCGCA |
| Hae II | RGCGCY |
| Hae III | GGCC |
| Hga I | GACGC |
| Hha I | GCGC |
| Hinc II | GTYRAC |
| Hind III | AAGCTT |
| Hinf I | GANTC |
| HinP1 I | GCGC |
| Hpa I | GTTAAC |
| Hpa II | CCGG |
| Hph I | GGTGA |
| Kas I | GGCGCC |
| Kpn I | GGTACC |
| Mdo I | GATC |
| Mbo II | GAAGA |
| Mfe I | CAATTG |
| Mlu I | ACGCGT |
| Mly I | GAGTCNNNNN |
| Mnl I | CCTC |
| Msc I | TGGCCA |
| Mse I | TTAA |
| Msl I | CAYNNNNRTG |
| MspA1 I | CMGCKG |
| Msp I | CCGG |
| Mwo I | GCNNNNNNNGC |
| Nae I | GCCGGC |
| Nar I | GGCGCC |
| Nci I | CCSGG |
| Nco I | CCATGG |
| Nde I | CATATG |
| NgoMI V | GCCGGC |
| Nbe I | GCTAGC |
| Nla III | CATG |
| Nla IV | GGNNCC |
| Not I | GCGGCCGC |
| Nru I | TCGCGA |
| Nsi I | ATGCAT |
| Nsp I | RCATGY |
| Pac I | TTAATTAA |
| PaeR7 I | CTCGAG |
| Pci I | ACATGT |
| PflF I | GACNNNGTC |
| PflM I | CCANNNNNTGG |
| Ple I | GAGTC |
| Pme I | GTTTAAAC |
| Pml I | CACGTG |
| PpuM I | RGGWCCY |
| PshA I | GACNNNNGTC |
| Psi I | TTATAA |
| PspG I | CCWGG |
| PspOM I | GGGCCC |
| Pst I | CTGCAG |
| Pvu I | CGATCG |
| Pvu II | CAGCTG |
| Rsa I | GTAC |
| Rsr II | CGGWCCG |
| Sac I | GAGCTC |
| Sac II | CCGCGG |
| Sal I | GTCGAC |
| Sap I | GCTCTTC |
| Sau3A I | GATC |
| Sau96 I | GGNCC |
| Sbf I | CCTGCAGG |
| Sca I | AGTACT |
| ScrF I | CCNGG |
| SexA I | ACCWGGT |
| SfaN I | GCATC |
| Sfc I | CTRYAG |
| Sfi I | GGCCNNNNNGGCC |
| Sfo I | GGCGCC |
| SgrA I | CRCCGGYG |
| Sma I | CCCGGG |
| Sml I | CTYRAG |
| SnaB I | TACGTA |
| Spe I | ACTAGT |
| Sph I | GCATGC |
| Ssp I | AATATT |
| Stu I | AGGCCT |
| Sty I | CCWWGG |
| Swa I | ATTTAAAT |
| Tag I | TCGA |
| Tfi I | GAWTC |
| Tli I | CTCGAG |
| Tse I | GCWGC |
| Tsp45 I | GTSAC |
| Tsp509 I | AATT |
| TspR I | CAGTG |
| Tth111 I | GACNNNGTC |
| Xba I | TCTAGA |
| Xca I | CCANNNNNNNNNTGG |
| Xho I | CTCGAG |
| Xma I | CCCGGG |
| Xmn I | GAANNNNTTC |

F. Methodologies

1. RNA Isolation and cDNA Synthesis

Total RNA is isolated using TRIZOL reagent (Gibco-BRL, Gaithersburg, Md.) following the manufacturer's instruction.

cDNA synthesis is carried out using SMART RACE cDNA Amplification Kit (Clontech, Palo Alto, Calif.) following manufacturer's instruction except that the specifically designed up-stream primer containing restriction enzyme sites for BmsF1 and BssK1 are used. The primer sequence is:

5′-AAGCAGTGGTAACAACGCAGGGACCGGG-3′.

2. Amplification

PCR: In PCR™, pairs of primers that selectively hybridize to nucleic acids are used under conditions that permit selective hybridization. The term primer, as used herein, encompasses any nucleic acid that is capable of priming the synthesis of a nascent nucleic acid in a template-dependent process. Primers may be provided in double-stranded or single-stranded form, although the single-stranded form is preferred.

The primers are used in any one of a number of template dependent processes to amplify the target-gene sequences present in a given template sample. One of the best known amplification methods is PCR™ which is described in detail in U.S. Pat. Nos. 4,683,195, 4,683,202 and 4,800,159, each incorporated herein by reference.

In PCR™, two primer sequences are prepared which are complementary to regions on opposite complementary strands of the target-gene(s) sequence. The primers will hybridize to form a nucleic-acid:primer complex if the target-gene(s) sequence is present in a sample. An excess of deoxyribonucleoside triphosphates are added to a reaction mixture along with a DNA polymerase, e.g., Taq polymerase, that facilitates template-dependent nucleic acid synthesis.

If the target-gene(s) sequence:primer complex has been formed, the polymerase will cause the primers to be extended along the target-gene(s) sequence by adding on nucleotides. By raising and lowering the temperature of the reaction mixture, the extended primers will dissociate from the target-gene(s) to form reaction products, excess primers will bind to the target-gene(s) and to the reaction products and the process is repeated. These multiple rounds of amplification, referred to as “cycles”, are conducted until a sufficient amount of amplification product is produced.

Next, the amplification product is detected. In certain applications, the detection may be performed by visual means. Alternatively, the detection may involve indirect identification of the product via fluorescent labels, chemiluminescence, radioactive scintigraphy of incorporated radiolabel or incorporation of labeled nucleotides, mass labels or even via a system using electrical or thermal impulse signals (Affymax technology).

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641, filed Dec. 21, 1990.

LCR: Another method for amplification is the ligase chain reaction (“LCR”), disclosed in European Patent Application No. 320,308, incorporated herein by reference. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

Qbeta Replicase: Qbeta Replicase, described in PCT Patent Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which can then be detected.

Isothermal Amplification: An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. Such an amplification method is described by Walker et al. 1992, incorporated herein by reference.

Strand Displacement Amplification: Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA.

Cyclic Probe Reaction: Target specific sequences can also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Transcription-Based Amplification: Other nucleic acid amplification procedures include transcription-based amplification systems (TAS), including nucleic acid sequence based amplification (NASBA) and 3SR, Kwoh et al. (1989); PCT Application WO 88/10315, 1989, each incorporated herein by reference).

In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Other Amplification Methods: Other amplification methods, as described in British Patent Application No. GB 2,202,328, and in PCT Application No. PCT/US89/01025, each incorporated herein by reference, may be used in accordance with the present invention. In the former application, “modified” primers are used in a PCR™ like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Davey et al., European Patent Application No. 329 822 (incorporated herein by reference) disclose a nucleic acid amplification process involving cyclically synthesizing single-stranded RNA (“ssRNA”), ssDNA, and double-stranded DNA (dsDNA), which may be used in accordance with the present invention.

The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Patent Application WO 89/06700 (incorporated herein by reference) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts.

Other suitable amplification methods include “race” and “one-sided PCR™” (Frohman, 1990; Ohara et al., 1989, each herein incorporated by reference). Methods based on ligation of two (or more) oligonucleotides in the presence of nucleic acid having the sequence of the resulting “di-oligonucleotide”, thereby amplifying the di-oligonucleotide, also may be used in the amplification step of the present invention, Wu et al., 1989, incorporated herein by reference).

3. Separation Methods

It normally is desirable, at one stage or another, to separate various products from reagents, such as the template or excess primers, or from other amplification products. In one embodiment, amplification products are separated by agarose, agarose-acrylamide or polyacrylamide gel electrophoresis using standard methods. See Sambrook et al., 1989. When working with nucleic acids, denaturing PAGE is preferred.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer, gas chromatography and HPLC (Freifelder, 1982).

Immobilization of the DNA may be achieved by a variety of methods involving either non-covalent or covalent interactions between the immobilized DNA comprising an anchorable moiety and an anchor. In a preferred embodiment of the invention immobilization consists of the non-covalent coating of a solid phase with streptavidin or avidin and the subsequent immobilization of a biotinylated polynucleotide (Holmstrom, 1993). It is further envisioned that immobilization may occur by precoating a polystyrene or glass solid phase with poly-L-Lys or poly L-Lys, Phe, followed by the covalent attachment of either amino- or sulfhydryl-modified polynucleotides using bifunctional crosslinking reagents.

Immobilization may also take place by the direct covalent attachment of short, 5′-phosphorylated primers to chemically modified polystyrene plates (“Covalink” plates, Nunc) Rasmussen, (1990). The covalent bond between the modified oligonucleotide and the solid phase surface is introduced by condensation with a water-soluble carbodiimide. This method facilitates a predominantly 5′-attachment of the oligonucleotides via their 5′-phosphates.

Nikiforov et al. (U.S. Pat. No. 5,610,287 incorporated herein by reference) describes a method of non-covalently immobilizing nucleic acid molecules in the presence of a salt or cationic detergent on a hydrophilic polystyrene solid support containing a hydrophilic moiety or on a glass solid support. The support is contacted with a solution having a pH of about 6 to about 8 containing the synthetic nucleic acid and a cationic detergent or salt. The support containing the immobilized nucleic acid may be washed with an aqueous solution containing a non-ionic detergent without removing the attached molecules.

Another commercially available method envisioned by the inventors to facilitate immobilization is the “Reacti-Bind.TM. DNA Coating Solutions” (see “Instructions—Reacti-Bind.TM. DNA Coating Solution” 1/1997). This product comprises a solution that is mixed with DNA and applied to surfaces such as polystyrene or polypropylene. After overnight incubation, the solution is removed, the surface washed with buffer and dried, after which it is ready for hybridization. It is envisioned that similar products, i.e. Costar “DNA-BIND™” or. Immobilon-AV Affinity Membrane (IAV, Millipore, Bedford, Mass.) are equally applicable to immobilize the respective fragment.

4. Blotting Techniques

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and detection is accomplished as described above.

5. Transformation Methods

Suitable methods for nucleic acid delivery for transformation of a cell for use with the current invention are believed to include virtually any method by which a nucleic acid (e.g., DNA) can be introduced into a cell, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome-mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection: In certain embodiments, a nucleic acid may be delivered to an organelle, a cell, a tissue or an organism via one or more injections (i.e., a needle injection), such as, for example, either subcutaneously, intradermally, intramuscularly, intervenously or intraperitoneally. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985).

Electroporation: In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in PCT Application No. WO 9217598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Calcium Phosphate: In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran: In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading: Additional embodiments of the present invention include the introduction of a nucleic acid by direct sonic loading. LTK⁻ fibroblasts have been transfected with the thymidine kinase gene by sonication loading (Fechheimer et al., 1987).

Liposome-Mediated Transfection: In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor-Mediated Transfection: Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al, 1990; Perales et al., 1994; Myers, EPO 0 273 085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

Microprojectile Bombardment: Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

Microprojectile bombardment may be used to transform various cell(s), tissue(s) or organism(s), such as for example any plant species. Examples of species which have been transformed by microprojectile bombardment include monocot species such as maize (PCT Application WO 95/06128), barley (Ritala et al., 1994; Hensgens et al., 1993), wheat (U.S. Pat. No. 5,563,055, incorporated herein by reference), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, incorporated herein by reference), sunflower (Knittel et al. 1994), peanut (Singsit et al., 1997), cotton (McCabe and Martinell, 1993), tomato (VanEck et al. 1995), and legumes in general (U.S. Pat. No. 5,563,055, incorporated herein by reference).

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

G. “Headless” Expression Vectors

Within certain embodiments, expression vectors are employed to express various polynucleotides in accordance with the present invention. Normally, expression vectors include all appropriate regulatory signals, including enhancers/promoters, transcription termination sites and poly-A signals. The present invention utilizes “headless” expression constructs that lack upstream promoter elements. They also include a selectable or screenable marker gene positioned between a 5′ cloning site and 3′ regulatory region. By inserting putative promoter sequences into this region, one may screen or select for promoter activity.

1. 5′ Regulatory Elements

As discussed above, “headless” expression constructs lack 5′ promoter elements. However, it may prove useful to have some regulatory signals, or proper sequence context. Thus, one may include elements that promoter mRNA stability, facilitate ribosome binding. In another embodiment, a “headless” expression construct will include and enhancer. Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

2. 3′ Regulatory Signals

Various 3′ regulatory signals may be included in the expression constructs of the present invention. For example, many eukaryotic transcripts conclude with a poly-A stretch. The precise function of these sequences is not known, but they likely contribute to transcript stability. Polyadenylation signals often utilized are those from SV40 and bovine or human growth hormone.

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (poly-A) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

3. Other Signals

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference.

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

4. Selectable and Screenable Markers

The expression constructs of the present invention will comprise a selectable or screenable marker gene. Such markers would confer an identifiable change to the cell permitting easy identification of cells containing a functioning expression construct. They also would permit, in the case of selectable markers, selection of specific clones containing promoters of interest.

Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed.

Screenable markers that may be employed include a β-glucuronidase (GUS) or uidA gene which encodes an enzyme for which various chromogenic substrates are known; an R-locus gene, which encodes a product that regulates the production of anthocyanin pigments (red color) in plant tissues (Dellaporta et al., 1988); a β-lactamase gene (Sutcliffe, 1978), which encodes an enzyme for which various chromogenic substrates are known (e.g., PADAC, a chromogenic cephalosporin); a xylE gene which encodes a catechol dioxygenase that can convert chromogenic catechols; an α-amylase gene (Ikuta et al., 1990); a green fluorescent protein (GFP) gene (Crameri et al., 1996) which encodes a protein that emits fluorescence upon excitation; a tyrosinase gene (Katz et al., 1983) which encodes an enzyme capable of oxidizing tyrosine to DOPA and dopaquinone which in turn condenses to form the easily-detectable compound melanin; a β-galactosidase gene, which encodes an enzyme for which there are chromogenic substrates; a luciferase (lux) gene, which allows for bioluminescence detection; an aequorin gene (Prasher et al., 1986) which may be employed in calcium-sensitive bioluminescence edetection; or a gene encoding for green fluorescent protein (Sheen et al., 1995; Haseloff et al., 1997; Reichel et al., 1996; WO 97/41228).

H. Library Production

In one embodiment, the present invention provides for the generation of a promoter library. Previous libraries have been obtained using random introduction of genomic DNA into vectors, followed by functional selection. This approach has various shortcomings, however, as described above. Thus, the present invention provides for an alternative approach that will facilitate a more complete promoter library reflective of most if not all functional promoter elements.

RNA, total or messenger, is extracted from a selected cell line or tissue. First strand cDNA synthesis is performed using oligo-dT primers as the downstream primer, and a primer containing a class III restriction enzyme site, a second restriction enzyme site 3′ to the class III site, and a poly-G at its 3′ end as the upstream primer. This upstream primer is biotinylated. Reverse transcriptase is added and used according to the manufacturer's instructions. Second strand synthesis is completed using any standard cDNA protocol.

The cDNA population thus produced is cut with a class III restriction enzyme that cleaves the upstream primer generated class III recognition site. Addition of streptavidin-coated magnetic beads, in one embodiment, permits the collection of the 5′-end fragment since the upstream primer introduced biotin into these molecules. Cleavage of the 5′-end fragment at the other primer generated site will release the 5′-end fragment from the beads; collection of the beads will remove unwanted sequences. What remains is a double-stranded fragment that contains a region corresponding to the 5′-end of the original transcript.

Taking the negative strand of the fragment produced in this fashion (obtained by a sequencing gel or HPLC), one can amplify the regions lying 5′ to the sequences corresponding to the fragment using a random primer to anchor the other end. If one uses a collection of 5′-end fragments (and random primers) to amplify genomic sequences, the resulting products can be cloned into vectors and constitute a promoter library. Particular constructs include those where the products are clone into a site upstream of a selectable or screenable marker, thereby facilitating identification of active promoters from other sequences.

I. Identification of Transcription Initiation Sites

As an additional aspect of the invention, it also is possible to use the disclosed methods to identify the general region in which transcription is initiated. Identification of the transcription initiation sites allows insight into the molecular basis of gene expression under a variety of conditions. Using the cDNA library of the invention as described above, and employing molecular techniques as is known to those skilled in the art, the general region of transcription initiation may be identified. The library may be obtained from a variety of tissues or cultured cells such as derived from a disease associated tissue.

From the cDNA library, the 5′ region containing the coding information may be isolated and sequenced as described herein. The 5′ transcript obtained may be compared with those of genomic sequences, for example GenBank sequences, in order to identify the transcription initiation sites. As is known to the skilled artisan the distribution of the initiation start site varies from gene to gene.

J. Identification of Transcription Factors

The present invention, in yet another aspect, provides for the identification of factors involved in transcriptional regulation. In developing a promoter library, it is possible to use this library as a target for transcription factors, including those previously unidentified. The production of a promoter library is described above. Once available, the promoter library can be used as follows.

A cDNA library is provided as a source for potential transcription factors. The library may be derived from a particular source, such as cardiac or tumor tissues, thereby biasing the identification of transcription factors towards those that are associated with particular tissues or disease states. The cDNA library is introduced into an appropriate host cell, e.g., yeast. The host cell contains, either integrated into its genome or episomally, a reporter plasmid that contains a promoter of interest—in the case of the present invention a promoter from the library described above. The reporter aspect is provided by a gene located downstream of the promoter. Such reporters are described elsewhere in this document.

When the proper combination of promoter and factor is provided within a single cell, the reporter gene should be transcribed and translated, and the product detected. The researcher then need only determine the identity of the transcription factor and, if unknown, the promoter. Unique factors may be identified, and known factors may be classified as transcription factors based on such interactions. In addition, the association of certain transcription factors with certain promoters or promoter elements also may be novel.

It is known that some transcription factors must work in conjunction with other factors in order to support transcription. Thus, in certain embodiments, it may also be important to provide expression or overexpression of other factors, such as TAF-II 31 and p300.

K Gene Expression Profiling

As discussed above, SAGE is a popular method for assessing gene expression in complex systems. SAGE, by nature, focuses on the 3′-end of transcripts. As such is it susceptible to the effects of Alu repeats, polyA tails, gene deletions and multiple promoters. The present invention provides for a different approach, designated a “TIPS assay,” where the 5′-end of the transcript is interrogated. In so doing, the shortcomings of SAGE can be avoided.

The initial steps of this protocol are the same as those outlined above for the generation of promoter libraries. At the step where the 5′-end fragment is blunted, the TIPS assay ligates the 5′-end fragment to another 5′-end fragment, thereby producing the TIPS version of a ditag—essentially two 5′-end fragment plus primer sequence. The ditag may then be amplified using PCR primer: 5′AAGCAGTGGTAACAACGCAGG-3′, cut with a restriction enzyme that will release the ditag, and concatenated for the purpose of cloning (TIPS library) and sequencing.

In this fashion, it is possible to assess a large number of different transcripts at the same time. Most importantly, it provides a TIPS library that is not biased against certain transcripts, as is the case with SAGE libraries.

L. Kits

All the essential materials and reagents required for performing reverse transcription, restriction, ligation, phosphorylation, phosphatasing, etc., may be assembled together in a kit. Such kits generally will comprise primers 1 and 2 (FIG. 1), and may also comprise polymerases (reverse transcriptases, DNA polymerases), restriction enzymes, ligase, dNTPs, buffers to provide the necessary reaction mixture for amplification, random primers, and in some cases, “headless” expression vectors, described above. All of the kits will provide suitable container means for storing and dispensing these reagents.

M. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

RNA Isolation and RT-PCR: Total RNA was isolated using TRI Reagent (MRC, Cincinnati, Ohio) following the manufacturer's instruction. The first strand cDNA was generated by reverse transcription. The reverse transcription was carried out in 20 μl reaction containing 1 μg total RNA, 1 μg oligdT₂₅ primer, 1 μg template switching primer (5′-AAG CAG TGG TAA CAA CGC AGG GAC CGG G-3′), 4 μl first-strand reaction buffer, 2 μl 10 mM dithiothreitol (DTT; Gibco-BRL, Gaithersburg, Md.), 1 μl 10 mM dNTPs, 1 μl SUPERase-in, (Amtion, Austin, Tex.) and 200 u Superscript II reverse transcriptase (Gibco-BRL). cDNA synthesis was incubated at 42° C. for 1 hour. After the first strand cDNA synthesis, 2 units of DNase free RNase (Ambion) was added to the reaction and followed by incubation at 37° C. for 15 min. Second strand cDNA synthesis was carried out by PCR in a 100 μl volume containing 2 μl of the RT reaction, 1 μl of dNTP solution (10 mM dATP, dTTP, dGTP and dCTP), 10 μl PCR reaction buffer, 10 μl 25 mM MgCl₂, 1 μl DNA polymerase (Roche, Branchburg, N.J.). The reaction was incubated at 95° C. for 10 min and then 15 cycles at: 95° C. 1 min., 65° C. 6 min. A prolonged elongation time up to 12 min was used in the final cycle. The PCR product was purified by QIAquick PCT Purification Kit (Qiagen, Valencia, Calif.).

Cleavage of PCR products with BsmF1 and Amplification of tags by PCR: Mix the reaction in a 100 μl solution containing 2 μl BSA (NEB), 10 μl 10×buffer (NEB), 10 μl BsmF1 and the PCR products. Incubate the reaction at 65° C. for one hour. Extract with equal volume of phenol/chloroform and ethanol precipitate. Use streptavidin coated beads to purify the cDNA fragments that contain the up-stream primer and 10 base pair nucleotide tag generated from the 5′-end of amplified cDNA. Blunt ends of the tags are generated using DNA polymerase I (Gibco, BRL) and linker added. The linker sequence is 5′CTGCTCGCGCCATCGATGGCGTTATTGTAATACGAC3′. The tags are amplified by PCR using the up-stream primer and the linker as the down-stream primer. After amplification, the products are digested by Bssk1 and the tags are purified using streptavidin coated beads. Run a 20% sequencing gel to separate the two strands of the tags and purify the anti-sense strand (the shorter one of the two). Use human genomic DNA as a template and the anti-sense strands as down-stream primers to generate genomic DNA product using Universal GenomeWalker Kit (Clontech). Purify the PCR products and clone those into a reporter vector, such as “TOPO Reporter Kit” (Invitrogen). The clones are sequenced for sequence analysis. The clones can be transfected into mammalian cells to detect the promoter function with the reporter gene activity as readout.

### REFERENCES

The following references, to the extent that they provide exemplary procedural or other details supplementary to those set forth herein, are specifically incorporated herein by reference.

U.S. Pat. No. 4,659,774

U.S. Pat. No. 4,683,195

U.S. Pat. No. 4,683,202

U.S. Pat. No. 4,684,611

U.S. Pat. No. 4,704,362

U.S. Pat. No. 4,800,159

U.S. Pat. No. 4,816,571

U.S. Pat. No. 4,883,750

U.S. Pat. No. 4,952,500

U.S. Pat. No. 4,959,463

U.S. Pat. No. 5,141,813

U.S. Pat. No. 5,221,619

U.S. Pat. No. 5,264,566

U.S. Pat. No. 5,302,523

U.S. Pat. No. 5,322,783

U.S. Pat. No. 5,384,253

U.S. Pat. No. 5,428,148

U.S. Pat. No. 5,464,765

U.S. Pat. No. 5,538,877

U.S. Pat. No. 5,538,880

U.S. Pat. No. 5,550,318

U.S. Pat. No. 5,554,744

U.S. Pat. No. 5,563,055

U.S. Pat. No. 5,574,146

U.S. Pat. No. 5,580,859

U.S. Pat. No. 5,583,013

U.S. Pat. No. 5,589,466

U.S. Pat. No. 5,602,244

U.S. Pat. No. 5,610,042

U.S. Pat. No. 5,610,287

U.S. Pat. No. 5,656,610

U.S. Pat. No. 5,702,932

U.S. Pat. No. 5,736,524

U.S. Pat. No. 5,780,448

U.S. Pat. No. 5,789,215

U.S. Pat. No. 5,866,330

U.S. Pat. No. 5,945,100

U.S. Pat. No. 5,981,274

U.S. Pat. No. 5,994,624

Adler and Modrich, “T7-induced DNA polymerase. Characterization of associated exonuclease activities and resolution into biologically active subunits,” J. Biol. Chem., 254:11605-11614, 1979.

Alberts et al., In: Molecular Biology of the Cell, Robertson (Ed.), Garland, N.Y., p 369, 1994.

Bates, “Genetic transformation of plants by protoplast electroporation,” Mol Biotechnol., 2(2):135-145, 1994.

Battraw and Hall, “Stable transformation of sorghum-bicolor protoplasts with chimeric neomycin phosphotransferase II and beta glucuronidase genes,” Theor. App. Genet., 82(2):161-168, 1991.

Beaucage, and Lyer, Tetrahedron, 48:2223-2311, 1992.

Bebenek and Kunkel, “The use of native T7 DNA polymerase for site-directed mutagenesis,” Nucl. Acids Res., 17:5408, 1989.

Bhattacharjee and Gupta, J. Plant Bioch. and Biotech. 6(2):69-73. 1997.

Boguski, “The turning point in genome research,” Trends Biochem. Sci., 21:295-296, 1995.

Bower et al., The Plant Journal, 2:409-416, 1992.

Buising and Benbow, “Molecular analysis of transgenic plants generated by microprojectile bombardment: effect of petunia transformation booster sequence,” Mol. Gen. Genet., 243:71-81, 1994.

Casas, Kononowicz, Zehr, Tomes, Axtell, Butler, Bressan, Hasegawa, “Transgenic sorghum plants via microprojectile bombardment,” Proc. Natl. Acad. Sci. USA, 90(23):11212-11216, 1993.

Chen and Okayama, “High-efficiency transformation of mammalian cells by plasmid DNA,” Mol. Cell. Biol. 7:2745-2752, 1987

Christou et al., Proc. Nat'l Acad Sci. USA, 84(12):3962-3966, 1987.

Cocea, “Duplication of a region in the multiple cloning site of a plasmid vector to enhance cloning-mediated addition of restriction sites to a DNA fragment,” Biotechniques, 23:814-816, 1997.

Crameri et al., Nat. Biotechnol 14, 315-319.

Dale et al, Plasmid, 13:31-40, 1985.

D'Alessio and Gerard, “Second-strand cDNA synthesis with E. coli DNA polymerase I and RNase H: the fate of information at the mRNA 5′ terminus and the effect of E. coli DNA ligase,” Nucl. Acids Res., 16:1999-2014, 1988.

Dellaporta et al., In: Chromosome Structure and Function: Impact of New Concepts, 18th Stadler Genetics Symposium, 11:263-282, 1988.

Derbyshire, Freemont, Sanderson, Beese, Friedman, Joyce, Steitz, “Genetic and crystallographic studies of the 3′,5′-exonucleolytic site of DNA polymerase I,” Science, 240:199-201, 1988.

DeRisi, Penland, Brown, Bittner, Meltzer, Ray, Chen, Su, “Use of a cDNA microarray to analyse gene expression patterns in human cancer,” Trent et al., Nat. Genet., 14(4):457-460, 1996.

D'Halluin et al., “Transgenic maize plants by tissue electroporation,” Plant Cell, 4(12):1495-1505, 1992.

Eckert and Kunkel, “DNA polymerase fidelity and the polymerase chain reaction,” PCR Methods and Applications, 1:17-24, 1991.

Engler, Lechner, Richardson, “Two forms of the DNA polymerase of bacteriophage T7,” J. Biol. Chem., 258:11165-11173, 1983.

EPA No. 320,308

EPA No. 329,822

EPO Application No. 0 273 085

Fechheimer, Boylan, Parker, Sisken, Patel and Zimmer, “Transfection of mammalian cells with plasmid DNA by scrape loading and sonication loading,” Proc Nat'l. Acad. Sci. USA 84:8463-8467, 1987

Fields, Adams, White, Venter, “How many genes in the human genome?,” Nat Genet, 7(3):345-346, 1994.

Fraley, Fomari, Kaplan, “Entrapment of a bacterial plasmid in phospholipid vesicles:potential for gene transfer,” Proc Nat'l. Acad. Sci. USA 76:3348-3352, 1979.

Freifelder, Physical Biochemistry Applications to Biochemistry and Molecular Biology, 2nd ed. Wm. Freeman and Co., New York, N.Y., 1982.

Frohman, In: PCR protocols: a guide to methods and applications, Academic Press, N.Y., 1990;

GB Patent App. 2,202,328

Gerhold and Caskey, “It's the genes! EST access to human genome contentm” BioEssays, 18:973-981, 1996.

Ghosh and Bachhawat, “Targeting of liposomes to hepatocytes,” In: Liver Diseases, Targeted Diagnosis and Therapy Using Specific Receptors and Ligands, Wu and Wu (Eds.), Marcel Dekker, New York, pp 87-104, 1991.

Gillam, Jahnke, Astell, Phillips, Hutchison, Smith, “Defined transversion mutations at a specific position in DNA using synthetic oligodeoxyribonucleotides as mutagens,” Nucleic Acids, Res, 6(9):2973-2973.

Gillam, Jahnke, Smith, “Enzymatic synthesis of oligodeoxyribonucleotides of defined sequence,” J. Biol. Chem., 253(8):2532-2539, 1978.

Gopal, “Gene transfer method for transient gene expression, stable transformation, and cotransformation of suspension cell cultures,” Mol. Cell. Biol. 5:1188-1190, 1985.

Graham and Van Der Eb, “A new technique for the assay of infectivity of human adenovirus 5 DNA,” Virology 52:456-467, 1973

Grippo and Richardson, “Deoxyribonucleic acid polymerase of bacteriophage T7,” J. Biol. Chem., 246:6867-6873, 1971.

Gubler and Hoffmann, “A simple and very efficient method for generating cDNA libraries,” Gene, 25:263-269, 1983.

Gubler, “Second-strand cDNA synthesis: mRNA fragments as primers,” Methods Enzymol., 152:330-335, 1987.

Harland and Weintraub, “Translation of mRNA injected into Xenopus oocytes is specifically inhibited by antisense RNA,” J. Cell Biol. 101:1094-1099, 1985.

Haseloff, Siemering, Prasher, Hodge, “Removal of a cryptic intron and subcellular localization of green fluorescent protein are required to mark transgenic Arabidopsis plants brightly,” Proc Natl Acad Sci USA, 94(6):2122-2127, 1997.

He et al., Plant Cell Reports, 14 (2-3): 192-196, 1994.

Hensgens et al., “Transient and stable expression of gusA fusions with rice genes in rice, barley and perennial ryegrass,” Plant Mol. Biol., 22(6): 1101-1127, 1993.

Holmstrom, Rossen, Rasmussen, “A highly sensitive and fast nonradioactive method for detection of polymerase chain reaction products,” Anal Biochem, 209(2):278-283, 1993.

Hori, Mark, Richardson, “Deoxyribonucleic acid polymerase of bacteriophage T7. Characterization of the exonuclease activities of the gene 5 protein and the reconstituted polymerase,” J. Biol. Chem., 254:11598-11604, 1979.

Hou and Lin, Plant Physiology, 111: 166, 1996.

Houts, Miyagi, Ellis, Beard, Beard, “Reverse transcriptase from avian myeloblastosis virus,” J. Virol., 29:517-522, 1979.

Hugh and Griffin, PCR Technology, 228-229, 1994.

Iiyy et al., Biotechnique 11:464, 1991.

Ikuta, Souza, Valencia, Castro, Schenberg, Pizzirani-Kleiner, Astolfi-Filho, “The alpha-amylase gene as a marker for gene cloning: direct screening of recombinant clones,” Biotechnology, 8(3):241-242, 1990.

Itakura and Riggs, “Chemical DNA synthesis and recombinant DNA studies,” Science 209:1401-1405, 1980.

Itakura K, Katagiri N, Narang S A, Bahl C P, Marians K J, Wu R, “Chemical synthesis and sequence studies of deoxyribooligonucleotides which constitute the duplex sequence of the lactose operator of Escherichia coli,” J Biol Chem, 250(12):4592-600, 1975.

Jannasch et al., Applied Environ. Microbiol., 58:3472-3481, 1992.

Kaeppler et al., Plant Cell Reports 9: 415-418, 1990.

Kaneda et al., “Introduction and expression of the human insulin gene in adult rat liver,” J Biol Chem., 264(21):12126-12129, 1989.

Kato et al., “Expression of hepatitis β virus surface antigen in adult rat liver. Co-introduction of DNA and nuclear protein by a simplified liposome method,” J. Biol. Chem., 266(6):3361-3364, 1991.

Katz, Thompson, Hopwood, “Cloning and expression of the tyrosinase gene from Streptomyces antibioticus in Streptomyces lividans,” J Gen Microbiol, 129(Pt 9):2703-14, 1983.

Khorana, “Total synthesis of a gene,” Science, 203(4381):614-25, 1979.

Klein et al., “High-velocity microprojectiles for delivering nucleic acids into living cells,” Nature, 327:70-73, 1987.

Knittel et al., Plant Cell Reports, 14(2-3):81-86, 1994.

Kong, Kucera, Jack, “Characterization of a DNA polymerase from the hyperthermophile archaea Thermococcus litoralis. Vent DNA polymerase, steady state kinetics, thermal stability, processivity, strand displacement, and exonuclease activities,” J. Biol. Chem., 268:1965-1975, 1993.

Kunkel, Roberts, Zakour, “Rapid and efficient site-specific mutagenesis without phenotypic selection,” Methods Enzymol., 154:367-382, 1987.

Kwoh, Davis, Whitfield, Chappelle, DiMichele, Gingeras, “Transcription-based amplification system and detection of amplified human immunodeficiency virus type 1 with a bead-based sandwich hybridization format,” Proc. Nat. Acad. Sci. USA, 86: 1173, 1989.

Lazzeri, “Stable transformation of barley via direct DNA uptake. Electroporation- and PEG-mediated protoplast transformation,” Methods Mol. Biol., 49:95-106, 1995.

Lee et al., Korean J. Genet., 11(2):65-72, 1989.

Levenson et al., “Internal ribosomal entry site-containing retroviral vectors with green fluorescent protein and drug resistance markers,” Human Gene Therapy, 9:1233-1236, 1998.

Lockhart, Dong, Byrne, Follettie, Gallo, Chee, Mittmann, Wang, Kobayashi, Horton, Brown, “Expression monitoring by hybridization to high-density oligonucleotide arrays,” Nature Biotech., 14:1675-1680, 1996.

Maniatis, Kee, Efstratiadis, Kafatos, “Amplification and characterization of a beta-globin gene synthesized in vitro,” Cell, 8(2):163-82, 1976.

Mattila, Korpela, Tenkanen, Pitkanen, “Fidelity of DNA synthesis by the Thermococcus litoralis DNA polymerase—an extremely heat stable enzyme with proofreading activity,” NAR, 19:4967-4973, 1991.

McCabe and Martinell, Bio-Technology, 11(5):596-598, 1993.

McClary, Ye, Hong, Witney, “Sequencing with the large fragment of DNA polymerase I from Bacillus stearothermophilus,” J. DNA Sequencing Mapping, 1(3):173-180, 1991.

Mead, McClary, Luckey, Kostichka, Witney, Smith, “Bst DNA polymerase permits rapid sequence analysis from nanogram amounts of template,” BioTechniques, 11(1):76-8, 80, 82-7, 1991.

Meinkoth and Wahl, “Nick translation,” Methods Enzymol, 152:91-94, 1987.

Modrich and Richardson, “Bacteriophage T7 deoxyribonucleic acid replication invitro. Bacteriophage T7 DNA polymerase: an an emzyme composed of phage- and host-specific subunits,” J. Biol. Chem., 250:5515-5522, 1975.

Murray and Kelley, “Characterization of lambdapolA transducing phages; effective expression of the E. coli polA gene,” Molec. Gen. Genet., 175:77-87, 1979.

Nicolau and Sene, “Liposome-mediated DNA transfer in eukaryotic cells: dependence of the transfer efficiency upon the type of liposomes used and the host cell cycle stage,” Biochem. Biophys. Acta, 721:185-190, 1982.

Nicolau et al, “Liposomes as carriers for in vivo gene transfer and expression,” Methods Enzymol., 149:157-176, 1987.

Nordstrom, Randahl, Slaby, Holmgren, “Characterization of bacteriophage T7 DNA polymerase purified to homogeneity by antithioredoxin immunoadsorbent chromatography,” J. Biol. Chem., 256:3112-3117, 1981.

Ohara, Dorit, Gilbert, “One-sided polymerase chain reaction: the amplification of cDNA,” Proc. Nat'l Acad. Sci. USA, 86: 5673-5677, 1989.

Omirulleh et al., “Activity of a chimeric promoter with the doubled CaMV ³⁵S enhancer element in protoplast-derived cells and transgenic plants in maize,” Plant Mol. Biol., 21:415-28, 1993.

PCT Patent App. 87/00880

PCT Patent App. 89/01025

Perler, Comb, Jack, Moran, Qiang, Kucera, Benner, Slatko, Nwankwo, Hempstead et al, “Intervening sequences in an Archaea DNA polymerase gene,” Proc. Nat'l Acad. Sci. USA, 89(12):5577-5581, 1992.

Perler, Kumar, Kong, “Thermostable DNA polymerases,” Adv. Protein Chem. 48:377-435, 1996

Potrykus et al., Mol. Gen. Genet., 199:183-188, 1985.

Potter et al, “Enhancer-dependent expression of human k immunoglobulin genes introduced into mouse pre-B lymphocytes by electroporation,” Proc Nat'l Acad. Sci. USA, 81:7161-7165, 1984.

Rasmussen, “Covalent immobilization of biomolecules onto polystyrene MicroWells for use in biospecific assays,” Ann Biol Clin, 48(9):647-50, 1990.

Reichel, Mathur, Eckes, Langenkemper, Koncz, Schell, Reiss, Maas, “Enhanced green fluorescence by the expression of an Aequorea victoria green fluorescent protein mutant in mono- and dicotyledonous plant cells,” Proc Natl Acad Sci USA, 93(12):5888-5893, 1996.

Rhodes et al, “Transformation of maize by electroporation of embryos,” Methods Mol. Biol., 55:121-131, 1995.

Rippe, Brenner and Leffert, “DNA-mediated gene transfer into adult rat hepatocytes in primary culture,” Mol. Cell Biol., 10:689-695, 1990.

Ritala et al., “Fertile transgenic barley to particle bombardment of immature embryos,” Plant Mol. Biol., 24(2):317-325, 1994.

Sambrook et al., In: Molecular Cloning: A Laboratory Manual, second edition, Cold Spring Harbor Laboratory, Cold Spring Harbor, N.Y., 1989.

Sanger, Nicklen, Coulson, “DNA sequencing with chain-terminating inhibitors,” Proc. Nat'l Acad. Sci. USA, 74:5463-5467, 1977.

Sheen, Hwang, Niwa, Kobayashi, Galbraith, “Green-fluorescent protein as a new vital marker in plant cells,” Plant J, 8(5):777-784, 1995.

Singsit et al., “Expression of a Bacillus thuringiensis cryIA(c) gene in transgenic peanut plants and its efficacy against lesser cornstalk borer,” Transgenic Res., 6:169-76, 1997.

Strausberg, Dahl and Klausner., “New opportunities for uncovering the molecular basis of cancer,” Nat. Genet., 15:415-416, 1997.

Studier, Rosenberg, Dunn, Dubendorff, “Use of T7 RNA polymerase to direct expression of cloned genes,” Methods Enzymol., 185:60-89, 1990.

Sutcliffe, “Nucleotide sequence of the ampicillin resistance gene of Escherichia coli plasmid pBR322,” Proc Natl Acad Sci USA, 75(8):3737-3741, 1978.

Szybalski, Universal restriction endonucleases: designing novel cleavage specificities by combining adapter oligodeoxynucleotide and enzyme moieties,” Gene, 40(2-3):169-173, 1985.

Tabor and Struhl, In: Current Protocols in Molecular Biology, Ausubel et al. (Eds.), John Wiley and Sons, NY, pp 3.5.10-3.5.12, 1989.

Tomes et al., “Transgenic tobacco plants and their progeny derived by microprojectile bombardment of tobacco leaves,” Plant Mol. Biol. 14:261-8, 1990.

Torbet et al., “Transformation of oat using mature embryo-derived tissue cultures,” Crop Science, 38:226-231, 1998.

Torbet et al., “Use of paromomycin as a selective agent for oat transformation,” Plant Cell Reports, 14:635-640, 1995.

Tsukada et al., Plant Cell Physiol., 30(4)599-604, 1989.

Tur-Kaspa, Teicher, Levine, Skoultchi and Shafritz, “Use of electroporation to introduce biologically active foreign genes into primary rat hepatocytes,” Mol. Cell Biol., 6:716-718, 1986.

Van Eck et al., Plant Cell Reports, 14(5):299-304, 1995.

Velculescu, Zhang, Vogelstein, Kinzler, “Serial analysis of gene expression,” Science, 270:484-487, 1995.

Wagner, Zenke, Cotten, Beug, Birnstiel, “Transferrin-polycation conjugates as carriers for DNA uptake into cells,” Proc. Nat'l Acad. Sci. USA 87(9):3410-3414, 1990.

Walker, Fraiser, Schram, Little, Nadeau, Malinowski, “Strand displacement amplification—an isothermal, in vitro DNA amplification technique,” Nucleic Acids Res, 20(7):1691-1696, 1992.

WO 88/10315

WO 89/06700

WO 90/07641

WO 92/17598

WO 94/09699

WO 95/06128

WO 97/41228

Wong et al., “Appearance of β-lactamase activity in animal cells upon liposome mediated gene transfer,” Gene, 10:87-94, 1980.

Wu and Wallace, “The ligation amplification reaction (LAR)—amplification of specific DNA sequences using sequential rounds of template-dependent ligation,” Genomics, 4:560-569, 1989.

Wu and Wu, Adv. Drug Delivery Rev., 12:159-167, 1993.

Yang, Burkholder, Roberts, Martinell and McCabe, “In vivo and in vitro gene transfer to mammalian somatic cells by particle bombardment,” Proc Nat'l Acad. Sci. USA, 87:9568-9572, 1990. Carbonelli, Corley, Seigelchifer, Zorzopulos, “A plasmid vector for isolation of strong promoters in E. coli,” FEMS Microbiol Lett. 177(1):75-82, 1999.

Zhang, Zhou, Velculescu, Kern, Hruban, Hamilton, Vogelstein, Kinzler, “Gene expression profiles in normal and cancer cells,” Science, 276:1268-1272, 1997.

Zhou, Broxmyer, Cooper, Harrington, and Srivastava “Adeno-associated virus 2 mediated gene transfer in murine hematopoietic cells, Exp. Hematol (N.Y.),

What is claimed is:

1. A method for generating a promoter library comprising:
(a) obtaining an RNA-containing composition from a cell;
(b) adding reverse transcriptase and a pair of primers to said composition, wherein said primers comprise
(i) an oligodT as a down-stream primer, and
(ii) a primer comprising three guanine residues at its 3-prime end as an up-stream primer, said primer also comprising a class II restriction enzyme site and a class III restriction enzyme site, wherein said class III site is 5′ to said class II site,
and incubating said primers and said reverse transcriptase under conditions supporting reverse transcription of a first corresponding cDNA strand and template switching by said reverse transcriptase;
(c) adding DNA polymerase to the product of step (b) under conditions supporting generation of a second corresponding cDNA;
(d) cleaving said cDNA population with a class III restriction enzyme that cleaves the up-stream primer generated class III restriction enzyme site;
(e) isolating the cDNA fragments lacking the poly-A tail of step (d), said fragments being designated as TIPS tags;
(f) ligating a linker to said TIPS tags;
(g) cleaving said TIPS tags+linkers with a class II restriction enzyme that cleaves the up-stream primer generated class II restriction site;
(h) obtaining the antisense strands from those portions of said TIP tags+linkers of step (g) that contain 5′ cDNA coding information;
(i) amplifying DNA sequences from genomic DNA using the antisense strands of step (h) and a random primer; and
(j) cloning the amplified products of step (i).

2. The method of claim 1, further comprising amplification of the cDNA prior to step (d).

3. The method of claim 2, wherein said amplification comprises DNA polymerase chain reaction.

4. The method of claim 1, wherein said class III restriction enzyme is a BsmF1.

5. The method of claim 4, wherein said class II restriction enzyme is selected from the group consisting of Hind III, EcoRI, SalI, BamHI and BssK I.

6. The method of claim 1, wherein said RNA composition is poly-A RNA.

7. The method of claim 1, wherein said up-stream primer further comprises a marker that permits isolation of said TIPS tags.

8. The method of claim 7, wherein said marker is binding ligand.

9. The method of claim 8, wherein said ligand is biotin.

10. The method of claim 1, wherein step (e) comprises binding of said biotin marker to streptavidin coated magnetic beads.

11. The method of claim 1, further comprising filling in the class III restriction enzyme site overhangs prior to step (f).

12. The method of claim 1, wherein step (j) comprises cloning said amplified products up-stream of a reporter coding region to create a promoter-reporter library.

13. The method of claim 12, wherein the reporter coding region is β-gal, luciferase or green fluorescent protein.

14. The method of claim 12, further comprising transforming a population of host cells with said promoter-reporter library.

15. The method of claim 14, wherein said host cells comprise bacteria cells.

16. The method of claim 15, further comprising screening the transformed bacteria cells for expression of said reporter.

17. The method of claim 16, further comprising sequencing expression positive clones.

18. The method of claim 1, further comprising cloning said TIPS tag, or a fragment thereof.

19. A method for identifying a transcription factor for a promoter comprising:
(a) obtaining an RNA-containing composition from a cell;
(b) adding reverse transcriptase and a pair of primers to said composition, wherein said primers comprise
(i) an oligodT as a down-stream primer, and
(ii) a primer comprising 3 guanine residues at its 3-prime end as an up-stream primer, said primer also comprising a class II restriction enzyme site and a class III restriction enzyme site, wherein said class III site is 5′ to said class II site, and incubating said primers and said reverse transcriptase under conditions supporting reverse transcription of a first corresponding cDNA strand and template switching by said reverse transcriptase;
(c) adding DNA polymerase to the product of step (b) under conditions supporting generation of a second corresponding cDNA;
(d) cleaving said cDNA population with a class III restriction enzyme that cleaves the up-stream primer generated class III restriction enzyme site;
(e) isolating the cDNA fragments lacking the poly-A tail of step (d), said fragments being designated as TIPS tags;
(f) ligating a linker to said TIPS tags;
(g) cleaving said TIPS tags+linkers with a class II restriction enzyme that cleaves the up-stream primer generated class II restriction site;
(h) obtaining the antisense strands from those portions of said TIP tags+linkers of step (g) that contain 5′ cDNA coding information;
(i) amplifying DNA sequences from genomic DNA using the antisense strands of step (h) and a random primer; and
(j) cloning the amplified products of step (i)
(k) sequencing the expression positive clones of step (j); and
(l) using the promoter identified in step (k) to identify a transcription factor acting thereon.

20. The method of claim 19, wherein step (l) comprises co-transformation, into a population of host cells, of:
(i) a construct comprising a reporter coding region under the control of a promoter identified in step (k); and
(ii) a construct comprising a cDNA expression vector,
wherein expression of said reporter in the presence of a given cDNA, but not in the absence of the same cDNA, indicates that said cDNA encodes a transcription factor that acts on said promoter.

21. The method of claim 20, wherein said host cell population comprises yeast cells.

22. The method of claim 20, further comprising sequencing of a cDNA found to encode a transcription factor.

23. The method of claim 20, wherein the cDNA expression construct is derived from the same organism as said promoter.

24. The method of claim 20, wherein the cDNA expression construct is derived from a different organism than said promoter.

25. A method for identifying the transcription initiation site of a gene comprising:
(a) obtaining an RNA-containing composition from a cell;
(b) adding reverse transcriptase and a pair of primers to said composition, wherein said primers comprise
(i) an oligodT as a down-stream primer, and
(ii) a primer comprising 3 guanine residues at its 3-prime end as an up-stream primer, said primer also comprising a class II restriction enzyme site and a class III restriction enzyme site, wherein said class III site is 5′ to said class II site, and incubating said primers and said reverse transcriptase under conditions supporting reverse transcription of a first corresponding cDNA strand and template switching by said reverse transcriptase;
(c) adding DNA polymerase to the product of step (b) under conditions supporting generation of a second corresponding cDNA;
(d) cleaving said cDNA population with a class III restriction enzyme that cleaves the up-stream primer generated class III restriction enzyme site;
(e) isolating the cDNA fragments lacking the poly-A tail of step (d), said fragments being designated as TIPS tags;
(f) ligating a linker to said TIPS tags, said linker comprising a primer sequence;
(g) cleaving said TIPS tags+linkers with a class II restriction enzyme that cleaves the up-stream primer generated class II restriction site;
(h) isolating that portion of said TIP tags+linkers of step (g) that contains 5′ cDNA coding information;
(i) treating the composition of step (h) with ligase to generate fragments that contain coding information from two different cDNAs, designated as DITags;
(j) cleaving said DITags of step (i) with said class II restriction enzyme that cleaves the up-stream primer generated class II restriction enzyme site, thereby releasing the DITtags;
(k) concatenating said DITtags;
(l) cloning said concatemers of step (k);
(m) sequencing the cloned concatemers of step (l); and
(n) comparing the sequence information of step (m) with at least one corresponding genomic sequence, thereby identifying the transcription start site of at least one corresponding mRNA.

26. The method of claim 25, further comprising amplification of the cDNA prior to step (d).

27. The method of claim 25, wherein said amplification comprises DNA polymerase chain reaction.

28. The method of claim 25, further comprising amplifying DITags prior to cleaving by said class II enzyme.

29. The method of claim 25, wherein said up-stream primer further comprises a marker that permits isolation of said TIPS tags.

30. The method of claim 29, wherein said marker is binding ligand.

31. The method of claim 30, wherein said ligand is biotin.

32. The method of claim 25, wherein step (e) comprises binding of said biotin marker to streptavidin coated magnetic beads.

33. The method of claim 25, further comprising filling in the class III restriction enzyme site overhangs generated by step (d).

34. The method of claim 25, wherein said class II restriction enzyme is selected from the group consisting of Hind III, EcoRI, SalI, BamHI and BssK I.

35. The method of claim 25, wherein said class III restriction enzyme is a BsmF1.

36. The method of claim 1, further comprising amplifying genomic sequences using a plurality of different primer sequences generated from sequence information obtained by sequencing of said TIPS tags.


SEQUENCE LISTING (3 sequences)

SEQ ID NO: 1 - DNA, 28 bp, Artificial Sequence
  Description of Artificial Sequence  Synthetic Primer
aagcagtggtaacaacgcagggaccggg

SEQ ID NO: 2 - DNA, 36 bp, Artificial Sequence
  Description of Artificial Sequence  Synthetic Primer
ctgctcgcgccatcgatggcgttattgtaatacgac

SEQ ID NO: 3 - DNA, 21 bp, Artificial Sequence
  Description of Artificial Sequence  Synthetic Primer
aagcagtggtaacaacgcagg